SEQUENCE LISTING (14 sequences)

SEQ ID NO: 1 - protein, 14 aa, Artificial Sequence
  Synthetically generated peptide
CCEYCCNPACTGCY

SEQ ID NO: 2 - protein, 14 aa, Artificial Sequence
  Synthetically generated sequence
CCEFCCNPACTGCY

SEQ ID NO: 3 - protein, 13 aa, Artificial Sequence
  Synthetically generated peptide
CCEFCCNPACTGC

SEQ ID NO: 4 - protein, 14 aa, Artificial Sequence
  Synthetically generated peptide
CCEICCNPACTGCY

SEQ ID NO: 5 - protein, 13 aa, Artificial Sequence
  Synthetically generated peptide
CCEICCNPACTGC

SEQ ID NO: 6 - protein, 14 aa, Artificial Sequence
  Synthetically generated peptide
CCELCCNPACTGCY

SEQ ID NO: 7 - protein, 13 aa, Artificial Sequence
  Synthetically generated peptide
CCELCCNPACTGC

SEQ ID NO: 8 - protein, 14 aa, Artificial Sequence
  Synthetically generated peptide
CCEWCCNPACTGCY

SEQ ID NO: 9 - protein, 13 aa, Artificial Sequence
  Synthetically generated peptide
CCEWCCNPACTGC

SEQ ID NO: 10 - protein, 13 aa, Artificial Sequence
  Synthetically generated peptide
CCEYCCNPACTGC

SEQ ID NO: 11 - protein, 15 aa, Artificial Sequence
  Synthetically generated peptide
PGTCEICAYAACTGC

SEQ ID NO: 12 - protein, 16 aa, Artificial Sequence
  Synthetically generated peptide
NDDCELCVNVACTGCL

SEQ ID NO: 13 - protein, 16 aa, Artificial Sequence
  Synthetically generated peptide
NDECELCVNVACTGCL

SEQ ID NO: 14 - protein, 14 aa, Artificial Sequence
  Synthetically generated sequence
CCEYCCNPACTGCY

(19) United States
(12) Patent Application Publication
(10) Pub. No.: US 2013/0045239 A1
(43) Pub. Date: Feb. 21, 2013

(54) Method for Modulating the Pharmacodynamic Effect of Orally Administered Guanylate Cyclase Receptor Agonists

(71) Applicants: Jeffrey Johnston, Boston, MA (US); Caroline Kurtz, Sudbury, MA (US)

(72) Inventors: Jeffrey Johnston, Boston, MA (US); Caroline Kurtz, Sudbury, MA (US)

(73) Assignee: Ironwood Pharmaceuticals, Inc., Cambridge, MA (US)

(21) Appl. No.: 13/390,123

(22) PCT Filed: Aug. 13, 2010
(86) PCT No.: PCT/US2010/045518

§ 371 Date: Sep. 28, 2012

Related U.S. Application Data
(60) Provisional application No. 61/233,740, filed on Aug. 13, 2009.

(51) Int. Cl. A61K 38/12; A61P 1/10; A61P 1/00; A61P 1/04; A61K 38/10; A61K 9/14

(52) U.S. Cl. 424/400; 514/21.5; 514/13.2; 514/21.1

(57) ABSTRACT

A method of modulating the pharmacodynamic effect of a GC-C receptor agonist polypeptide formulation in a subject in need of such treatment is disclosed, The method comprises administering the GC-C receptor agonist polypeptide formulation to the subject before the ingestion of food.

### PRIORITY CLAIM

This application claims priority to U.S. Application Ser. No. 61/233,740, filed Aug. 13, 2009. The entire contents of the aforementioned application are incorporated herein by reference.

### FIELD

This disclosure concerns methods of modulating the pharmacodynamic effect of a GC-C receptor agonist polypeptide formulations in a subject in need of such treatment.

### SEQUENCE LISTING

This application incorporates by reference in its entirety the Sequence Listing entitled “mod_effect_app_ST25.txt” (4 kilobytes) which was created Aug. 13, 2010 and filed electronically herewith.

### BACKGROUND OF THE INVENTION

Linaclotide, a polypeptide having the amino acid sequence Cys Cys Glu Tyr Cys Cys Asn Pro Ala Cys Thr Gly Cys Tyr (SEQ ID NO: 1), activates the guanylate cyclase-C (GC-C) receptor (See, e.g., U.S. Pat. No. 7,304,036 and U.S. Pat. No. 7,371,727).

Linaclotide and other GC-C receptor agonists (such as those disclosed in any of U.S. Pat. No. 7,304,036, U.S. Pat. No. 7,371,727, WO 02/78683, WO 2004/069165, WO2005/087797, WO 2007/022531, WO2005/016244, WO2005/074575, WO2006/102069, WO2008/002971, WO2008/106429, WO 2008/137318, WO2002/078683, WO 2006/086653, WO 2007/101158, WO 2008/151257, U.S. Pat. No. 7,041,786, and WO 2007/101161) may be administered orally for the treatment of gastrointestinal disorders and conditions including irritable bowel syndrome (IBS) and chronic constipation (CC). Solid formulations comprising linaclotide have been developed for oral administration.

Methods are needed for modulating the pharmacodynamic effect of these linaclotide formulations as well as other GC-C receptor agonist polypeptide formulations.

### SUMMARY OF THE INVENTION

These and other needs are met by the present invention, which provides a method for decreasing the pharmacodynamic effect of a GC-C receptor agonist polypeptide formulation which is administered to a subject in need of such treatment, comprising administering the GC-C receptor agonist polypeptide to the subject before the ingestion of food.

In some embodiments, the GC-C receptor agonist polypeptide is administered to the subject as a formulation which comprising the GC-C receptor agonist polypeptide, a pharmaceutically acceptable carrier, and one or more agents selected from a cation selected from Mg²⁺, Ca²⁺, Zn²⁺, Mn²⁺, K⁺, Na⁺ and Al³⁺ and a sterically hindered primary amine.

In another embodiment, the invention also provides a method of decreasing the pharmacodynamic effect of linaclotide which is administered to a subject in need of such treatment, comprising administering linaclotide to the subject before the ingestion of food.

The invention also provides a method of decreasing the pharmacodynamic effect of a linaclotide formulation which is administered to a subject in need of such treatment, comprising administering the GC-C receptor agonist polypeptide formulation to the subject before the ingestion of food, wherein the GC-C receptor agonist formulation comprises linaclotide, a pharmaceutically acceptable carrier, and one or more agents selected from a cation selected from Mg²⁺, Ca²⁺, Zn²⁺, Mn²⁺, K⁺, Na⁺ and Al³⁺ and a sterically hindered primary amine.

The invention also provides a method of decreasing the pharmacodynamic effect of a GC-C receptor agonist polypeptide formulation which is administered to a subject suffering from irritable bowel syndrome or constipation, comprising administering the GC-C receptor agonist polypeptide formulation to the subject before the ingestion of food, wherein the GC-C receptor agonist formulation comprises a GC-C receptor agonist polypeptide, a pharmaceutically acceptable carrier, Ca⁺², and leucine.

The invention also provides a method of decreasing the pharmacodynamic effect of a linaclotide formulation which is administered to a subject suffering from irritable bowel syndrome (e.g., constipation-predominant irritable bowel syndrome) or constipation (e.g., chronic constipation), comprising administering the linaclotide formulation to the subject before the ingestion of food, wherein the GC-C receptor agonist formulation comprises linaclotide or a pharmaceutically acceptable salt of linaclotide, a pharmaceutically acceptable carrier, Ca⁺², and leucine.

The invention also provides a method of decreasing the pharmacodynamic effect of a linaclotide formulation which is administered to a subject suffering from irritable bowel syndrome or constipation, comprising administering the linaclotide formulation to the subject before the ingestion of food, wherein the linaclotide agonist formulation is in the form of a tablet or capsule that comprises:

(a) Linaclotide;

(b) CaCl₂.2H₂O;

(c) L-Leucine; and

(d) Hydroxypropyl Methylcellulose

wherein linaclotide is present in the pharmaceutical composition in an amount between 100 μg to 600 μg and the molar ratio of Ca²⁺:leucine:linaclotide is between 5-100:5-50:1.

In some embodiments, linaclotide is present in the tablet or capsule in an amount of 133 or 266 μg. In some embodiments, CaCl₂ is present in the tablet or capsule in an amount of 1541 μg. In some embodiments, leucine is present in the tablet or capsule in an amount of 687 μg. In some embodiments, hydroxypropyl methylcellulose is present in the tablet or capsule in an amount of 700 μg.

The invention also provides a method of decreasing the pharmacodynamic effect of a linaclotide formulation which is administered to a subject suffering from irritable bowel syndrome or constipation, comprising administering the linaclotide formulation to the subject before the ingestion of food, wherein the linaclotide agonist formulation is in the form of a tablet or capsule that comprises:

(a) Linaclotide;

(b) CaCl₂.2H₂O;

(c) L-Leucine; and

(d) Hydroxypropyl Methylcellulose

wherein linaclotide is present in the pharmaceutical composition in an amount between 100 μg to 600 μg and the molar ratio of Ca²⁺:leucine:linaclotide is between 5-100:5-50:1.

More particularly, linaclotide is present in the tablet or capsule in an amount of 133 or 266 μg.

More particularly, CaCl₂ is present in the tablet or capsule in an amount of 1541 μg.

More particularly, leucine is present in the tablet or capsule in an amount of 687 μg.

More particularly, hydroxypropyl methylcellulose is present in the tablet or capsule in an amount of 700 μg.

The invention also provides a method of treating irritable bowel syndrome or constipation in a subject in need of such treatment, comprising: administering a GC-C receptor agonist polypeptide to the subject before the ingestion of food.

### DETAILED DESCRIPTION OF THE INVENTION

### Definitions

As used herein, the term “binder” refers to any pharmaceutically acceptable binder that may be used in the practice of the invention. Examples of pharmaceutically acceptable binders include, without limitation, a starch (e.g., corn starch, potato starch and pre-gelatinized starch (e.g., STARCH 1500® and STARCH 1500 LM®, sold by Colorcon, Ltd.) and other starches), maltodextrin, gelatin, natural and synthetic gums such as acacia, powdered tragacanth, guar gum, cellulose and its derivatives (e.g., methylcellulose, hydroxyethyl cellulose, hydroxyethyl methylcellulose, hydroxypropyl cellulose and hydroxypropyl methylcellulose (hypromellose), ethyl cellulose, cellulose acetate, carboxymethyl cellulose calcium, sodium carboxymethyl cellulose, carboxymethylcellulose, microcrystalline cellulose (e.g. AVICEL™, such as, AVICEL-PH-101™, -103™ and -105™, sold by FMC Corporation, Marcus Hook, Pa., USA)), polyvinyl alcohol, polyvinyl pyrrolidone (e.g., polyvinyl pyrrolidone K30), and mixtures thereof.

As used herein, the term “filler” refers to any pharmaceutically acceptable filler that may be used in the practice of the invention. Examples of pharmaceutically acceptable fillers include, without limitation, talc, calcium carbonate (e.g., granules or powder), dibasic calcium phosphate, tribasic calcium phosphate, calcium sulfate (e.g., granules or powder), microcrystalline cellulose (e.g., Avicel PH101 or Celphere CP-305), powdered cellulose, dextrates, kaolin, mannitol, silicic acid, sorbitol, starch (e.g., Starch 1500), pre-gelatinized starch, lactose, glucose, fructose, galactose, trehalose, sucrose, maltose, isomalt, raffinose, maltitol, melezitose, stachyose, lactitol, palatinite, xylitol, myoinositol, and mixtures thereof.

Examples of pharmaceutically acceptable fillers that may be particularly used for coating with litnaclotide include, without limitation, talc, microcrystalline cellulose (e.g., Avicel PH 101 or Celphere CP-305), powdered cellulose, dextrates, kaolin, mannitol, silicic acid, sorbitol, starch, pre-gelatinized starch, lactose, glucose, fructose, galactose, trehalose, sucrose, maltose, isomalt, dibasic calcium phosphate, raffinose, maltitol, melezitose, stachyose, lactitol, palatinite, xylitol, mannitol, myoinositol, and mixtures thereof.

As used herein, the term “additives” refers to any pharmaceutically acceptable additive. Pharmaceutically acceptable additives include, without limitation, disintegrants, dispersing additives, lubricants, glidants, antioxidants, coating additives, diluents, surfactants, flavoring additives, humectants, absorption promoting additives, controlled release additives, anti-caking additives, anti-microbial agents (e.g., preservatives), colorants, desiccants, plasticizers and dyes.

As used herein, an “excipient” is any pharmaceutically acceptable additive, filler, binder or agent.

As used herein, the term “alkyl”, as used herein, refers to a saturated linear or branched-chain monovalent hydrocarbon radical. Unless otherwise specified, an alkyl group contains 1-20 carbon atoms (e.g., 1-20 carbon atoms, 1-10 carbon atoms, 1-8 carbon atoms, 1-6 carbon atoms, 1-4 carbon atoms or 1-3 carbon atoms). Examples of alkyl groups include, but are not limited to, methyl, ethyl, n-propyl, isopropyl, n-butyl, isobutyl, s-butyl, t-butyl, pentyl, hexyl, heptyl, octyl and the like.

As used herein, the terms Cₙ₋ₘ “alkoxyalkyl” and Cₙ₋ₘ “thioalkoxyalkyl” mean alkyl, substituted with one or more alkoxy or thioalkoxy groups, as the case may be, wherein the combined total number of carbons of the alkyl and alkoxy groups, or alkyl and thioalkoxy groups, combined, as the case may be, is between the values of n and m. For example, a C₄₋₆ alkoxyalkyl has a total of 4-6 carbons divided between the alkyl and alkoxy portion; e.g. it can be —CH₂OCH₂CH₂CH₃, —CH₂CH₂OCH₂CH₃ or —C₁₋₂CH₂CH₂OCH₃.

As used herein, the term “aryl” (as in “aryl ring” or “aryl group”), used alone or as part of a larger moiety, refers to a carbocyclic ring system wherein at least one ring in the system is aromatic and has a single point of attachment to the rest of the molecule. Unless otherwise specified, an aryl group may be monocyclic, bicyclic or tricyclic and contain 6-18 ring members. Examples of aryl rings include, but are not limited to, phenyl, naphthyl, indanyl, indenyl, tetralin, fluorenyl, and anthracenyl.

As used herein, the term “heteroaryl” (or “heteroaromatic” or “heteroaryl group” or “aromatic heterocycle”) used alone or as part of a larger moiety as in “heteroaralkyl” or “heteroarylalkoxy” refers to a ring system wherein at least one ring in the system is aromatic and contains one or more heteroatoms, wherein each ring in the system contains 3 to 7 ring members and which has a single point of attachment to the rest of the molecule. Unless otherwise specified, a heteroaryl ring system may be monocyclic, bicyclic or tricyclic and have a total of five to fourteen ring members. In one embodiment, all rings in a heteroaryl system are aromatic. Also included in this definition are heteroaryl radicals where the heteroaryl ring is fused with one or more aromatic or non-aromatic carbocyclic or heterocyclic rings, or combinations thereof, as long as the radical or point of attachment is in the heteroaryl ring. Bicyclic 6,5 heteroaromatic system, as used herein, for example, is a six membered heteroaromatic ring fused to a second five membered ring wherein the radical or point of attachment is on the six membered ring.

Heteroaryl rings include, but are not limited to the following monocycles: 2-furanyl, 3-furanyl, N-imidazolyl, 2-imidazolyl, 4-imidazolyl, 5-imidazolyl, 3-isoxazolyl, 4-isoxazolyl, 5-isoxazolyl, 2-oxazolyl, 4-oxazolyl, 5-oxazolyl, N-pyrrolyl, 2-pyrrolyl, 3-pyrrolyl, 2-pyridyl, 3-pyridyl, 4-pyridyl, 2-pyrimidinyl, 4-pyrimidinyl, 5-pyrimidinyl, pyridazinyl (e.g., 3-pyridazinyl), 2-thiazolyl, 4-thiazolyl, 5-thiazolyl, tetrazolyl (e.g., 5-tetrazolyl), triazolyl (e.g., 2-triazolyl and 5-triazolyl), 2-thienyl, 3-thienyl, pyrazolyl (e.g., 2-pyrazolyl), isothiazolyl, 1,2,3-oxadiazolyl, 1,2,5-oxadiazolyl, 1,2,4-oxadiazolyl, 1,2,3-triazolyl, 1,2,3-thiadiazolyl, 1,3,4-thiadiazolyl, 1,2,5-thiadiazolyl, pyrazinyl, 1,3,5-triazinyl, and the following bicycles: benzimidazolyl, benzofuryl, benzothiophenyl, benzopyrazinyl, benzopyranonyl, indolyl (e.g., 2-indolyl), purinyl, quinolinyl (e.g., 2-quinolinyl, 3-quinolinyl, 4-quinolinyl), and isoquinolinyl (e.g., 1-isoquinolinyl, 3-isoquinolinyl, or 4-isoquinolinyl).

### Stable GC-C Receptor Agonist Polypeptide Formulations

The formulations used in the method contain a GC-C receptor agonist polypeptide such as linaclotide, a pharmaceutically acceptable salt thereof, or a polypeptide as disclosed in any of U.S. Pat. No. 7,304,036, U.S. Pat. No. 7,371,727, WO 02/78683, WO 2004/069165, WO2005/087797, WO 2007/022531, WO2005/016244, WO2005/074575, WO2006/102069, WO2008/002971, WO2008/106429, WO 2008/137318, WO2002/078683, WO 2006/086653, WO 2007/101158, WO 2008/151257, U.S. Pat. No. 7,041,786, and WO 2007/101161.

The solid, stable formulations used in the invention contain a GC-C receptor agonist polypeptide as described in any of the above documents or linaclotide or a pharmaceutically acceptable salt of linaclotide. The formulations are stable and have a sufficient shelf life for manufacturing, storing and distributing the drug. For example, the formulations have an expected shelf life of at least 12 months at room temperature storage conditions (e.g., 25° C./60 percent relative humidity (RH)) and up to at least 18 months or 24 months at room temperature storage conditions (e.g., 25° C./60 percent RH). In the formulations, greater than or equal to 95 percent of the original amount of linaclotide in the composition remains after three months when packaged samples are stored at accelerated conditions (40° C./75 percent RH) when assessed in an assay on a weight/weight basis as determined by high pressure liquid chromatography (HPLC) against a linaclotide reference standard.

The GC-C receptor agonist polypeptide formulations are prepared from a solution, e.g., an aqueous solution (“the coating solution”), comprising: (i) a GC-C receptor agonist polypeptide such as linaclotide or a pharmaceutically acceptable salt thereof; (ii) a cation selected from Mg²⁺, Ca²⁺, Zn²⁺, Mn²⁺, K⁺, Na⁺ and Al³⁺ and/or a sterically hindered primary amine (e.g., leucine); and optionally (iii) a pharmaceutically acceptable binder. The GC-C receptor agonist polypeptide formulations can optionally include one or more of: a pharmaceutically acceptable glidant, a pharmaceutically acceptable lubricant or a pharmaceutically acceptable additive that acts as both a glidant and lubricant.

It has been found that a cation selected from Mg²⁺, Ca²⁺, Zn²⁺, Mn²⁺, K⁺, Na⁺ and Al³⁺ is useful for suppressing the formation of an oxidation product of the GC-C receptor agonist polypeptide linaclotide during storage. It has also been found that a sterically hindered primary amine is useful for suppressing the formation of a formaldehyde imine adduct of the GC-C receptor agonist polypeptide linaclotide (“formaldehyde imine product”) during storage. Thus, the GC-C receptor agonist polypeptide formulations comprising a cation selected from Mg²⁺, Ca²⁺, Zn²⁺, Mn²⁺, K⁺, Na⁺ or Al³⁺—that is, a divalent cation selected from Zn²⁺, Mg²⁺ and Ca²⁺—and/or a sterically hindered primary amine, such as an amino acid, have a sufficient shelf life (as measured by chromatographic purity and/or by a weight/weight assay) for manufacturing, storing and distributing the drug. Further, while the presence of a sterically hindered amine alone can increase the formation of a hydrolysis product of linaclotide during storage, the combination of a sterically hindered primary amine and a cation, e.g., the combination of leucine and Ca²⁺, suppresses the formation of the hydrolysis product of the GC-C receptor agonist polypeptide as well as the oxidation product of GC-C receptor agonist polypeptide during storage, leading to an even greater overall stability as determined by a weight/weight assay and/or by chromatographic purity.

GC-C receptor agonist polypeptide formulations are typically produced as follows.

### Preparation of the Coating Solution:

Approximately 32 g to 42 g of purified water is mixed with hydrochloric acid to create a solution with a pH between 1.5 and 2.0. The cation, if used, is added to the solution in a quantity to provide the desired concentration, and the solution is mixed for sufficient time to produce a clear solution. The sterically hindered primary amine, if used, is added to the solution in a quantity to provide the desired concentration, and the solution is mixed for sufficient time to produce a clear solution. Other additives, such as antioxidants, are then added, if desired. The pH of the solution is tested, and hydrochloric acid is added, if necessary, to produce a solution having a pH between 1.5 and 2.0. The binder is then added to the solution and the mixture is then stirred for sufficient time to achieve a clear solution. The desired amount of linaclotide is added to the solution and mixed for 30-100 minutes to provide the coating solution.

Preparation of the Active Beads:

Approximately 30-36 g of dried microcrystalline cellulose beads are added to a Mini Column Fluid Bed Coater. The microcrystalline cellulose beads are fluidized and heated prior to layering. Next, the coating solution is layered to the beads. The spraying temperature is controlled between 24° C. and 55° C. by controlling inlet temperature, spray rate, atomization pressure, and air volume. After the entire coating solution is layered to the beads, the beads are dried. The product of this process is referred to as active beads.

Preparation of Active Beads with Protective Coating (Optional):

Approximately 35 g of Active Beads are added to a Mini Column Fluid Bed Coater. The Active Beads are fluidized and heated prior to coating with Aquacoat (e.g. Aquacoat Ethylcellulose Aquaeous Dispersion, 15% w/w, FMC Biopolymer, ECD-30), Eudragit (e.g. Eudragit E PO PE-EL, Roehm Pharma Polymers) or Opadry (e.g Opadry AMB dispersion, 20% w/w, Colorcon). Next, the coating solution is layered to the beads. The spraying temperature is controlled between 24° C. and 55° C. by controlling inlet temperature, spray rate, atomization pressure, and air volume. After the entire coating solution is layered to the beads, the beads are dried.

### Formulation Scheme B

Preparation of the Coating Solution:

Approximately 8.3 kg of purified water is mixed with hydrochloric acid to create a solution with a pH between 1.5 and 2.0. The cation, if used, is added to the solution in a quantity to provide the desired concentration, and the solution is mixed for sufficient time to produce a clear solution. The sterically hindered primary amine, if used, is added to the solution in a quantity to provide the desired concentration, and the solution is mixed for sufficient time to produce a clear solution. Other additives, such as antioxidants, are then added, if desired. The binder is then added to the solution and the solution is mixed for sufficient time to achieve a clear solution. The pH of the solution is tested, and hydrochloric acid is added if necessary to produce a solution having a pH between 1.5 and 2.0. This is Solution 1. Approximately 8.3 kg of purified water is mixed with hydrochloric acid to create a solution with a pH between 1.5 and 2.0. The desired amount of linaclotide is added to the solution and mixed for 10 to 30 minutes. The pH of the solution is tested, and hydrochloric acid is added if necessary to produce a solution having a pH between 1.5 and 2.0. This is Solution 2. Solution 1 and Solution 2 are then mixed together. The pH of the solution is tested, and hydrochloric acid is added if necessary to produce a solution having a pH between 1.5 and 2.0. This is the coating solution.

Preparation of the Active Beads:

Approximately 24.19 kg of microcrystalline cellulose beads are added to a Wurster Column of a Glatt GPCG-30 Fluid Bed. The microcrystalline cellulose beads are fluidized and heated to product temperature of 45-47° C. Next, the coating solution is layered to the beads. The product spraying temperature is controlled between 37° C. and 47° C. by controlling inlet temperature, spray rate, atomization pressure, and air volume. After the entire coating solution is layered to the beads, the beads are dried with a product drying temperature of 37° C. to 47° C. The product of this process is referred to as active beads.

### Indications

The GC-C receptor agonist polypeptide formulations can be used to treat a variety of disorders in patients. Typically, the patient is suffering from: a disorder selected from the group consisting of gastrointestinal motility disorders, chronic intestinal pseudo-obstruction, colonic pseudo-obstruction, duodenogastric reflux, dyspepsia, functional dyspepsia, nonulcer dyspepsia, a functional gastrointestinal disorder, functional heartburn, gastroesophageal reflux disease (GERD), gastroparesis, irritable bowel syndrome (e.g., constipation-predominant irritable bowel syndrome (c-IBS) and/or alternating irritable bowel syndrome (a-IBS)), post-operative ileus, chronic constipation, constipation, pain associated with constipation, and disorders and conditions associated with constipation (e.g., constipation associated with use of opiate pain killers, post-surgical constipation, and constipation associated with neuropathic disorders as well as other conditions and disorders described herein); and colonic pseudo-obstruction. In a further embodiment, the patient has been diagnosed with irritable bowel syndrome (e.g. (e.g. diarrhea predominant-IBS, constipation predominant-IBS, and/or alternating-IBS), according to the Rome Criteria (e.g. Rome II).

The dose range of the GC-C receptor agonist polypeptide (specifically linaclotide) for adult humans is generally from 25 μg to 6 mg per day orally. In one embodiment, the dose range is 25 μg to 2 mg per day orally of linaclotide. In a further embodiment, the dose range for adult humans is 50 μg to 1 mg per day orally of linaclotide (e.g., 50 μg, 67.5 μg, 100 μg, 133 μg, 150 μg, 200 μg, 250 μg, 266 μg, 300 μg, 350 μg, 400 μg, 450 μg, 500 μg, 550 μg, 600 μg, 650 μg, 700 μg, 750 μg, 800 μg, 850 μg, 900 μg, 950 μg or 1 mg). In yet a further embodiment, the dose range is 100 μg to 600 μg per day orally of linaclotide. In other embodiments, the dose is 50 μg, 67.5 μg, 100 μg, 133 μg, 150 μg, 200 μg, 266 μg, 300 μg, 400 μg, 500 μg or 600 μg linaclotide per day orally.

### Methods

In one embodiment, the invention provides a method of decreasing the pharmacodynamic effect of a GC-C receptor agonist polypeptide formulation which is administered to a subject in need of such treatment, comprising administering the GC-C receptor agonist polypeptide formulation to the subject before the ingestion of food. In a further embodiment, the GC-C receptor agonist formulation comprises a GC-C receptor agonist polypeptide, a pharmaceutically acceptable carrier, and one or more agents selected from a cation selected from Mg²⁺, Ca²⁺, Zn²⁺, Mn²⁺, K⁺, Na⁺ or Al³⁺ and a sterically hindered primary amine. In yet a further embodiment, the GC-C receptor agonist polypeptide is linaclotide.

It has been found that the pharmacodynamic effect of a GC-C receptor agonist polypeptide (e.g., linaclotide) may be modulated by administering the polypeptide either before the ingestion of food or with food (e.g., with a meal or soon after ingesting a meal). Thus, the pharmacodynamic effect of the GC-C receptor agonist polypeptide (e.g., linaclotide) may be adjusted according the therapeutic needs of the subject in a beneficial manner, e.g., the pharmacodynamic effect may be modulated to improve one or more therapeutic indices or outcomes or, to decrease one or more undesired outcomes in a subject. Specifically, it has been found that administering a GC-C receptor agonist polypeptide (e.g., linaclotide) before the ingestion of food decreases the pharmacodynamic effect of the polypeptide, thus decreasing the risk of potential side effects (e.g., loose stools or diarrhea). Conversely, one may administer a GC-C receptor agonist polypeptide to increase the pharmacodynamic effect of the polypeptide if a greater therapeutic effect is desired

“Before the ingestion of food” means prior to eating; that is, 15 minutes, 30 minutes, 45 minutes, 60 minutes, 2 hours, 4, hours, 6 hours 8 hours, 10 hours, 12 hours, and up to 24 hours before the ingestion of food. Administration of the formulation prior to the consumption of food decreases the pharmacodynamic effect of the GC-C receptor agonist polypeptide while minimizing potential adverse events. These adverse events may include, for example, loose stools. “Before the ingestion of food” also means the GC-C receptor agonist polypeptide formulation is administered on an empty stomach.

Thus, in one aspect, the GC-C receptor agonist polypeptide formulation is administered 15 minutes to 4 hours before the ingestion of food.

In another aspect, the GC-C receptor agonist polypeptide formulation is administered to the subject from 15 minutes to 24 hours before the ingestion of food.

In another aspect, the GC-C receptor agonist polypeptide formulation is administered at least 15 minutes before the ingestion of food.

In another aspect, the GC-C receptor agonist polypeptide formulation is administered to the subject 30 minutes to 8 hours before the ingestion of food.

In another aspect, the GC-C receptor agonist polypeptide formulation is administered to the subject 30 minutes before the ingestion of food.

In another aspect, the GC-C receptor agonist polypeptide formulation is administered to the subject at least 30 minutes before the ingestion of food.

Thus, in another aspect, the GC-C receptor agonist polypeptide formulation is administered to the subject 45 minutes before the ingestion of food.

Thus, in another aspect, the GC-C receptor agonist polypeptide formulation is administered to the subject 60 minutes before the ingestion of food.

In another aspect, the GC-C receptor agonist polypeptide formulation is administered to the subject 30 minutes to 8 hours before the ingestion of food.

In another aspect, the GC-C receptor agonist polypeptide formulation is administered to the subject 1 hour to 18 hours before the ingestion of food.

In another aspect, the GC-C receptor agonist polypeptide formulation is administered to the subject 4 to 12 hours before the ingestion of food.

In another aspect, the GC-C receptor agonist polypeptide formulation is administered to the subject 1 to 8 hours before the ingestion of food.

In another aspect, the GC-C receptor agonist polypeptide formulation is administered to a subject having an empty stomach.

In a further embodiment of the above aspects, the GC-C receptor agonist polypeptide is linaclotide or a pharmaceutically acceptable salt thereof.

In a further aspect, the pharmacodynamic effect is measured by the Bristol Stool Form Scale (BSFS), the number of spontaneous bowel movements (SBM) in a given time period and/or the number of complete SBM (CSBM) in a given time period. A decrease in the pharmacodynamic effect may be measured by a decrease in the BSFS, SBM or CSBM when the GC-C receptor agonist polypeptide formulation is administered to a subject before the ingestion of food (e.g., at least 15 minutes, at least 30 minutes, at least 45 minutes, at least one hour or at least two hours before the ingestion of food or at least four hours, at least eight hours, at least ten hours or at least 12 hours after a prior ingestion of food) compared to the pharmacodynamic effect of the GC-C receptor agonist polypeptide formulation when it is administered to a subject with food (e.g., a meal) or shortly after ingestion of food (e.g., within 15 minutes, within 30 minutes, within 90 minutes or within two hours after ingestion of food).

In another aspect, the pharmacodynamic effect results in a lower score on the Bristol Stool Form Scale (BSFS) when the formulation is administered to the subject before ingestion of food as compared to when the formulation is administered to the subject with food or shortly after ingestion of food.

In another aspect, the pharmacodynamic effect results in fewer spontaneous bowel movements (SBM) in a time period (e.g., a 24 hour period, three day time period, seven day time period, two week time period or four week time period) when the formulation is administered to the subject before ingestion of food as compared to when the formulation is administered to the subject with food or shortly after ingestion of food.

In another aspect, the pharmacodynamic effect results in fewer spontaneous bowel movements in a time period (e.g., a 24 hour period) when the formulation is administered to a subject before ingestion of food as compared to when the formulation is administered to the subject with food or shortly after ingestion of food.

In another aspect, the pharmacodynamic effect results in fewer complete spontaneous bowel movements (CSBM) in a time period (e.g., a 24 hour period, three day time period, seven day time period, two week time period or four week time period) when the formulation is administered to the subject before ingestion of food as compared to when the formulation is administered to the subject with food or shortly after ingestion of food.

In another aspect, the pharmacodynamic effect results in a lower score on the Bristol Stool Form Scale (BSFS), fewer spontaneous bowel movements (SBM) in a time period (e.g., a 24 hour period, three day time period, seven day time period, two week time period or four week time period), and fewer complete spontaneous bowel movements (CSBM) in a time period (e.g., a 24 hour period, three day time period, seven day time period, two week time period or four week time period) when the formulation is administered to the subject before ingestion of food as compared to when the formulation is administered to the subject with food or shortly after ingestion of food.

In another aspect, the pharmacodynamic effect results in a lower score on the Bristol Stool Form Scale (BSFS) and fewer spontaneous bowel movements (SBM) in a time period (e.g., a 24 hour period, three day time period, seven day time period, two week time period or four week time period), when the formulation is administered to a subject who has not eaten as compared to said subject before ingestion of food as compared to when the formulation is administered to the subject with food or shortly after ingestion of food.

In another aspect, the pharmacodynamic effect results in a lower score on the Bristol Stool Form Scale (BSFS) and fewer complete spontaneous bowel movements (CSBM) in a time period (e.g., a 24 hour period, three day time period, seven day time period, two week time period or four week time period), when the formulation is administered to a subject before ingestion of food as compared to when the formulation is administered to the subject with food or shortly after ingestion of food.

In another aspect, the pharmacodynamic effect results in fewer SBM in a time period (e.g., a 24 hour period, three day time period, seven day time period, two week time period or four week time period) and fewer CSBM in a time period (e.g., a 24 hour period, three day time period, seven day time period, two week time period or four week time period) when the formulation is administered to a subject before ingestion of food as compared to when the formulation is administered to the subject with food or shortly after ingestion of food.

As indicated previously, the GC-C receptor agonist polypeptide formulation of the invention method comprises a GC-C receptor agonist polypeptide, a pharmaceutically acceptable carrier, and one or more agents selected from a cation and a sterically hindered primary amine.

In one aspect, the GC-C receptor agonist polypeptide is selected from linaclotide and any of the polypeptides disclosed in any of U.S. Pat. No. 7,304,036, U.S. Pat. No. 7,371,727, WO 02/78683, WO 2004/069165, WO2005/087797, WO 2007/022531, WO2005/016244, WO2005/074575, WO2006/102069, WO2008/002971, WO2008/106429, WO 2008/137318, WO2002/078683, WO 2006/086653, WO 2007/101158, WO 2008/151257, U.S. Pat. No. 7,041,786, and WO 2007/101161.

More particularly, the polypeptide is selected from the group consisting of:

(SEQ ID NO: 2)
CCEFCCNPACTGCY,
(SEQ ID NO: 3)
CCEFCCNPACTGC,
(SEQ ID NO: 4)
CCEICCNPACTGCY,
(SEQ ID NO: 5)
CCEICCNPACTGC,
(SEQ ID NO: 6)
CCELCCNPACTGCY,
(SEQ ID NO: 7)
CCELCCNPACTGC,
(SEQ ID NO: 8)
CCEWCCNPACTGCY,
(SEQ ID NO: 9)
CCEWCCNPACTGC,
(SEQ ID NO: 10)
CCEYCCNPACTGC,
(SEQ ID NO: 11)
PGTCEICAYAACTGC,
(SEQ ID NO: 12)
NDDCELCVNVACTGCL,
(SEQ ID NO: 13)
NDECELCVNVACTGCL,
and
(SEQ ID NO: 14)
CCEYCCNPACTGCY.

More particularly, the GC-C receptor agonist polypeptide is linaclotide.

In another aspect, the agent is cation selected from the group consisting of Mg²⁺, Ca²⁺, Zn²⁺, Mn²⁺, K⁺, Na⁺ and Al³⁺.

In another aspect, the Mg²⁺, Ca²⁺, Zn²⁺, Mn²⁺, K⁺, Na⁺ or Al³⁺ is provided as magnesium acetate, magnesium chloride, magnesium phosphate, magnesium sulfate, calcium acetate, calcium chloride, calcium phosphate, calcium sulfate, zinc acetate, zinc chloride, zinc phosphate, zinc sulfate, manganese acetate, manganese chloride, manganese phosphate, manganese sulfate, potassium acetate, potassium chloride, potassium phosphate, potassium sulfate, sodium acetate, sodium chloride, sodium phosphate, sodium sulfate, aluminum acetate, aluminum chloride, aluminum phosphate or aluminum sulfate.

More particularly, the Mg²⁺, Ca²⁺, Zn²⁺, Mn²⁺, K⁺, Na⁺ or Al³⁺ is provided as magnesium chloride, calcium chloride, calcium phosphate, calcium sulfate, zinc acetate, manganese chloride, potassium chloride, sodium chloride or aluminum chloride.

In another aspect, the agent is Mg²⁺, Ca²⁺ or Zn²⁺.

More particularly, the Mg²⁺, Ca²⁺ or Zn²⁺ is provided as magnesium chloride, calcium chloride or zinc acetate.

More particularly, the agent is Ca²⁺.

More particularly, the Ca²⁺ is provided as calcium chloride.

In another aspect, the agent is a sterically hindered primary amine.

More particularly, the sterically hindered primary amine is an amino acid.

More particularly, the amino acid is a naturally-occurring amino acid.

More particularly, the naturally-occurring amino acid is histidine, phenylalanine, alanine, glutamic acid, aspartic acid, glutamine, leucine, methionine, asparagine, tyrosine, threonine, isoleucine, tryptophan, glycine or valine.

More particularly, the naturally-occurring amino acid is leucine, isoleucine, alanine or methionine.

More particularly, the naturally-occurring amino acid is leucine or methionine.

More particularly, the naturally-occurring amino acid is leucine.

Alternatively, the sterically hindered primary amine is a non-naturally occurring amino acid.

More particularly, the non-naturally occurring amino acid is 1-aminocyclohexane carboxylic acid.

Alternatively, the sterically hindered primary amine has the formula: wherein R₁, R₂ and R₃ are independently selected from: H; —C(O)OH; C₁-C₆ alkyl, optionally substituted by —CO₂H, —CONH₂, or a 5-10 membered aryl or heteroaryl; C₁-C₆ alkoxyalkyl; or C₁-C₆ thioalkoxyalkyl, wherein any of the alkyl or aryl groups above can be singly or multiply substituted with halogen or —NH₂, and provided that no more than two of R₁, R₂ and R₃ are H. In a further embodiment, no more than one of R₁, R₂ and R₃ is H.

More particularly, the sterically hindered primary amine has the formula:

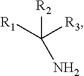

wherein R₁, R₂ and R₃ are independently selected from: H; —C(O)OH; C₁-C₆ alkyl; C₁-C₆ alkoxyalkyl; or C₁-C₆ thioalkoxyalkyl, wherein any of the alkyl or aryl groups above can be singly or multiply substituted with halogen or —NH₂, and provided that no more than two of R₁, R₂ and R₃ are H. In a further embodiment, no more than one of R₁, R₂ and R₃ is H.

More particularly, the sterically hindered primary amine has the formula:

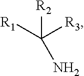

wherein R₁, R₂ and R₃ are independently selected from: H; —C(O)OH; C₁-C₆ alkyl; C₁-C₆ alkoxyalkyl; or C₁-C₆ thioalkoxyalkyl, and provided that no more than two of R₁, R₂ and R₃ are H. In a further embodiment, no more than one of R₁, R₂ and R₃ is H.

In another embodiment, the sterically hindered primary amine is cyclohexylamine or 2-methylbutylamine.

In another embodiment, the sterically hindered primary amine is chitosan.

In any of these aspects and embodiments, the GC-C receptor agonist polypeptide may be linaclotide.

In another aspect, the GC-C receptor agonist polypeptide formulation used in the method comprises a cation selected from Mg²⁺, Ca²⁺, Zn²⁺, Mn²⁺, K⁺, Na⁺ and Al³⁺ and a sterically hindered primary amine.

More particularly, the Mg²⁺, Ca²⁺, Zn²⁺, Mn²⁺, K⁺, Na⁺ or Al³⁺ is provided as magnesium acetate, magnesium chloride, magnesium phosphate, magnesium sulfate, calcium acetate, calcium chloride, calcium phosphate, calcium sulfate, zinc acetate, zinc chloride, zinc phosphate, zinc sulfate, manganese acetate, manganese chloride, manganese phosphate, manganese sulfate, potassium acetate, potassium chloride, potassium phosphate, potassium sulfate, sodium acetate, sodium chloride, sodium phosphate, sodium sulfate, aluminum acetate, aluminum chloride, aluminum phosphate or aluminum sulfate.

More particularly, the Mg²⁺, Ca²⁺, Zn²⁺, Mn²⁺, K⁺, Na⁺ or Al³⁺ is provided as magnesium chloride, calcium chloride, calcium phosphate, calcium sulfate, zinc acetate, manganese chloride, potassium chloride, sodium chloride or aluminum chloride.

More particularly, the cation is selected from Mg²⁺, Ca²⁺ or Zn²⁺.

More particularly, the Mg²⁺, Ca²⁺ or Zn²⁺ is provided as magnesium chloride, calcium chloride or zinc acetate.

More particularly, the cation is Ca²⁺.

More particularly, the Ca²⁺ is provided as calcium chloride.

More particularly, the sterically hindered primary amine is an amino acid.

More particularly, the amino acid is a naturally-occurring amino acid.

More particularly, the naturally-occurring amino acid is histidine, phenylalanine, alanine, glutamic acid, aspartic acid, glutamine, leucine, methionine, asparagine, tyrosine, threonine, isoleucine, tryptophan, glycine or valine.

More particularly, the naturally-occurring amino acid is leucine, isoleucine, alanine or methionine.

More particularly, the naturally-occurring amino acid is leucine or methionine.

More particularly, the naturally-occurring amino acid is leucine.

Alternatively, the sterically hindered primary amine is a non-naturally occurring amino acid.

More particularly, the non-naturally occurring amino acid is 1-aminocyclohexane carboxylic acid.

Alternatively, the sterically hindered primary amine has the formula: wherein R₁, R₂ and R₃ are independently selected from: H; —C(O)OH; C₁-C₆ alkyl, optionally substituted by —CO₂H, —CONH₂, or a 5-10 membered aryl or heteroaryl; C₁-C₆ alkoxyalkyl; or C₁-C₆ thioalkoxyalkyl, wherein any of the alkyl or aryl groups above can be singly or multiply substituted with halogen or —NH₂, and provided that no more than two of R₁, R₂ and R₃ are H. In a further embodiment, no more than one of R₁, R₂ and R₃ is H.

More particularly, the sterically hindered primary amine has the formula:

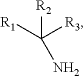

wherein R₁, R₂ and R₃ are independently selected from: H; —C(O)OH; C₁-C₆ alkyl; C₁-C₆ alkoxyalkyl; or C₁-C₆ thioalkoxyalkyl, wherein any of the alkyl or aryl groups above can be singly or multiply substituted with halogen or —NH₂, and provided that no more than two of R₁, R₂ and R₃ are H. In a further embodiment, no more than one of R₁, R₂ and R₃ is H.

More particularly, the sterically hindered primary amine has the formula:

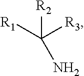

wherein R₁, R₂ and R₃ are independently selected from: H; —C(O)OH; C₁-C₆ alkyl; C₁-C₆ alkoxyalkyl; or C₁-C₆ thioalkoxyalkyl, and provided that no more than two of R₁, R₂ and R₃ are H. In a further embodiment, no more than one of R₁, R₂ and R₃ is H.

In another embodiment, the sterically hindered primary amine is cyclohexylamine or 2-methylbutylamine.

In another embodiment, the sterically hindered primary amine is chitosan.

In any of these aspects or embodiments, the GC-C receptor agonist polypeptide may be linaclotide.

In these aspects, the GC-C receptor agonist polypeptide formulation additionally comprises a pharmaceutically acceptable binder. In particular, the pharmaceutically acceptable binder is selected from polyvinyl alcohol, polyvinylpyrrolidone (povidone), a starch, maltodextrin or a cellulose ether. More particularly, the pharmaceutically acceptable binder is a cellulose ether which may be selected from: methylcellulose, ethylcellulose, carboxymethylcellulose, hydroxyethyl cellulose, hydroxyethyl methylcellulose, hydroxypropyl cellulose and hydroxypropyl methylcellulose.

In these aspects, the GC-C receptor agonist polypeptide formulation additionally comprises a pharmaceutically acceptable glidant, lubricant or additive that acts as both a glidant and lubricant.

In these aspects, the GC-C receptor agonist polypeptide formulation additionally comprises an antioxidant. In particular, the antioxidant is BHA, vitamin E or propyl gallate.

In these aspects, the GC-C receptor agonist polypeptide formulation additionally comprises a pharmaceutically acceptable filler. In particular, the pharmaceutically acceptable filler is cellulose, isomalt, mannitol or dibasic calcium phosphate.

More particularly, the cellulose used in the filler is selected from microfine cellulose and microcrystalline cellulose. More particularly, the pharmaceutically acceptable filler comprises particles having an average diameter between 150 μm and 1000 μm.

In another aspect, the sterically hindered primary amine is leucine and the cation is Ca²⁺.

In another aspect, the molar ratio of Ca²⁺ to leucine is at least 1:1.

More particularly, the molar ratio of Ca²⁺ to leucine is at least 1.5:1.

More particularly, the molar ratio of Ca²⁺ to leucine is at least 2:1.

In this aspect, the sterically hindered amine is leucine and the molar ratio of leucine to GC-C receptor agonist polypeptide is at least 10:1.

More particularly, the molar ratio of leucine to GC-C receptor agonist polypeptide is at least 20:1.

More particularly, the molar ratio of leucine to GC-C receptor agonist polypeptide in the pharmaceutical composition is at least 30:1.

In the GC-C receptor agonist polypeptide formulations comprising a filler, the weight ratio of GC-C receptor agonist polypeptide to pharmaceutically acceptable filler is between 1:25 and 1:2,500.

More particularly, the weight ratio of GC-C receptor agonist polypeptide to pharmaceutically acceptable filler is between 1:100 and 1:2000.

More particularly, the weight ratio of GC-C receptor agonist polypeptide to pharmaceutically acceptable filler is between 1:100 and 1:1000.

In the GC-C receptor agonist polypeptide formulations, comprising a cation and a sterically hindered primary amine, the molar ratio of cation:sterically hindered primary amine:GC-C receptor agonist polypeptide is 40-100:20-50:1.

More particularly, when the cation is Ca²⁺ and the sterically hindered primary amine is leucine, the molar ratio of Ca²⁺:leucine:GC-C receptor agonist polypeptide is 100:30:1, 80:40:1, 80:30:1, 80:20:1, 60:30:1, 60:20:1, 50:30:1, 50:20:1, 40:20:1, 20:20:1, 10:10:1, 10:5:1, 5:10:1 or 5:5:1.

More particularly, the molar ratio of Ca²⁺:leucine:GC-C receptor agonist polypeptide is 60:30:1.

More particularly, the cation is provided as CaCl₂.

In any of these aspects or embodiments, the GC-C receptor agonist polypeptide may be linaclotide.

In another aspect, the GC-C receptor antagonist formulation is in the form of a capsule or tablet.

In particular, each capsule or tablet comprises 50 μg to 1 mg GC-C receptor agonist polypeptide.

More particularly, each capsule or tablet comprises 100 μg, 150 μg, 200 μg, 300 μg, 400 μg, 500 μg or 600 μg GC-C receptor agonist polypeptide.

In some embodiments, each capsule or tablet comprises 50 μg to 1 mg linaclotide. In some embodiments, each capsule or tablet comprises 100 μg, 133 μg, 150 μg, 200 μg, 266 μg, 300 μg, 400 μg, 500 μg or 600 μg linaclotide.

In another aspect, the subject in need of such treatment is suffering from a disorder selected from the group consisting of gastrointestinal motility disorder, chronic intestinal pseudo-obstruction, colonic pseudo-obstruction, duodenogastric reflux, dyspepsia, functional dyspepsia, nonulcer dyspepsia, functional gastrointestinal disorder, functional heartburn, gastroesophageal reflux disease (GERD), gastroparesis, irritable bowel syndrome, post-operative ileus, and constipation.

In another aspect, the subject in need of treatment is suffering from irritable bowel syndrome with constipation (IBS-c) or alternating IBS (IBS-a).

In another aspect, the subject in need of treatment is suffering from irritable bowel syndrome with constipation (IBS-c). In this aspect, a once daily effective amount of the pharmaceutical formulation described herein is administered to the patient. In various aspects, the pharmaceutical formulation comprises 50 μg to 1 mg linaclotide (more particularly, 100 μg, 133 μg, 150 μg, 200 μg, 266 μg, 300 μg, 400 μg, 500 μg or 600 μg linaclotide; even more particularly 133 μg or 266 μg linaclotide) or another GC-C receptor agonist polypeptide per unit dose per day. In other aspects, the pharmaceutical composition is administered for a period of at least one day, two days, three days, four days, five days, six days, one week, two weeks, three weeks, four weeks, twelve weeks or longer. In some aspects, treatment with the linaclotide composition improves at least one symptom selected from reduced abdominal pain, an increase in the number of complete spontaneous bowel movements (CSBM) in a week, an increase in the number of spontaneous bowel movements (SBM) in a week, improved stool consistency, reduced straining, reduced abdominal discomfort, reduced bloating or reduced IBS-c symptom severity.

In another aspect, the subject in need of such treatment is suffering from constipation (e.g., chronic constipation). In this aspect, a once daily effective amount of the pharmaceutical formulation described herein is administered to the patient. In various aspects, the pharmaceutical formulation comprises 50 μg to 1 mg linaclotide (more particularly, 100 μg, 133 μg, 150 μg, 200 μg, 266 μg, 300 μg, 400 μg, 500 μg or 600 μg linaclotide; even more particularly 133 μg or 266 μg linaclotide) or another GC-C receptor agonist polypeptide per unit dose per day. In other aspects, the pharmaceutical composition is administered for a period of at least one day, two days, three days, four days, five days, six days, one week, two weeks, three weeks, four weeks, twelve weeks or longer. In some embodiments, treatment with the linaclotide composition improves at least one symptom selected from an increase in the number of complete spontaneous bowel movements (CSBM) in a week, an increase in the number of spontaneous bowel movements (SBM) in a week, improved stool consistency, reduced straining, reduced abdominal discomfort, reduced bloating or reduced severity of constipation.

Stool consistency of each bowel movement (BM) may be monitored by the 7-point Bristol Stool Form Scale (BSFS) (1=hard lumps, 2=lumpy sausage, 3=cracked sausage, 4=smooth sausage, 5=soft lumps, 6=mushy, 7=watery). Straining may be monitored by the 7-point Ease of Passage Scale (1=manual disimpaction/enema needed, 2=severe straining, 3=moderate straining, 4=mild straining, 5=no straining, 6=urgency, 7=incontinent). CSBM may be measured by the sensation of complete emptying after an SBM (yes/no). Abdominal discomfort, bloating and severity of constipation may be measured using, e.g., a 5-point ordinal scale (1=none, 2=mild, 3=moderate, 4=severe, 5=very severe).

In another embodiment, the invention provides a method of decreasing the pharmacodynamic effect of a GC-C receptor agonist polypeptide which is administered to a subject in need of such treatment, comprising administering the GC-C receptor agonist polypeptide to the subject before the ingestion of food.

In one aspect of this embodiment, the GC-C receptor agonist polypeptide is administered to the subject 15 minutes to 4 hours before the ingestion of food.

In another aspect of this embodiment, the GC-C receptor agonist polypeptide is administered to the subject 1 to 18 hours before the ingestion of food.

In another aspect of this embodiment, the GC-C receptor agonist polypeptide is administered to the subject 4 to 12 hours before the ingestion of food.

In another aspect of this embodiment, the GC-C receptor agonist polypeptide is administered to the subject 30 minutes to 8 hours before the ingestion of food.

In another aspect of this embodiment, the GC-C receptor agonist polypeptide is administered to a subject having an empty stomach.

In another aspect of this embodiment, the GC-C receptor agonist polypeptide formulation is administered to the subject at least 15 minutes before the ingestion of food.

In another aspect of this embodiment, the GC-C receptor agonist polypeptide formulation is administered to the subject at least 30 minutes before the ingestion of food.

In another aspect of this embodiment, the GC-C receptor agonist polypeptide formulation is administered to the subject at least 1 hour before the ingestion of food.

In another aspect of this embodiment, the GC-C receptor agonist polypeptide formulation is administered to the subject at least 2 hours before the ingestion of food.

In another embodiment, the invention provides a method of increasing the pharmacodynamic effect of a GC-C receptor agonist polypeptide which is administered to a subject in need of such treatment, comprising administering the GC-C receptor agonist polypeptide to the subject after a prior ingestion of food. In one embodiment, the GC-C receptor agonist polypeptide is administered at least four hours, at least eight hours, at least ten hours or at least 12 hours after a prior ingestion of food. In a further embodiment, the GC-C receptor agonist polypeptide is administered on an empty stomach.

In any of these aspects or embodiments, the GC-C receptor agonist polypeptide may be linaclotide.

In another aspect of this embodiment, the GC-C receptor agonist polypeptide is administered as a formulation comprising the GC-C receptor agonist polypeptide and a pharmaceutically acceptable excipient. In some embodiments, the GC-C receptor agonist polypeptide is linaclotide.

In another aspect of this embodiment, the subject in need of such treatment is suffering from a disorder selected from the group consisting of irritable bowel syndrome (IBS) and constipation. In one aspect, the disorder is IBS, which is constipation-predominant IBS (IBS-c) or alternating IBS (IBS-a). More particularly, the disorder is IBS-c. In another aspect, the subject in need of such treatment is suffering from constipation. More particularly, the disorder is constipation which is chronic constipation, idiopathic constipation, post-operative ileus, or constipation caused by opiate use.

In another aspect of this embodiment, the formulation further comprises one or more agents selected from a cation selected from Mg²⁺, Ca²⁺, Zn²⁺, Mn²⁺, K⁺, Na⁺ and Al³⁺ or a sterically hindered primary amine.

In particular, the Mg²⁺, Ca²⁺, Zn²⁺, Mn²⁺, K⁺, Na⁺ or Al³⁺ is provided as magnesium acetate, magnesium chloride, magnesium phosphate, magnesium sulfate, calcium acetate, calcium chloride, calcium phosphate, calcium sulfate, zinc acetate, zinc chloride, zinc phosphate, zinc sulfate, manganese acetate, manganese chloride, manganese phosphate, manganese sulfate, potassium acetate, potassium chloride, potassium phosphate, potassium sulfate, sodium acetate, sodium chloride, sodium phosphate, sodium sulfate, aluminum acetate, aluminum chloride, aluminum phosphate or aluminum sulfate.

More particularly, the Mg²⁺, Ca²⁺, Zn²⁺, Mn²⁺, K⁺, Na⁺ or Al³⁺ is provided as magnesium chloride, calcium chloride, calcium phosphate, calcium sulfate, zinc acetate, manganese chloride, potassium chloride, sodium chloride or aluminum chloride.

More particularly, the cation is Mg²⁺, Ca² or Zn²⁺.

More particularly, the Mg²⁺, Ca²⁺ or Zn²⁺ is provided as magnesium chloride, calcium chloride or zinc acetate.

More particularly, the agent is Ca²⁺.

More particularly, the Ca²⁺ is provided as calcium chloride.

Alternatively, the agent is a sterically hindered primary amine.

More particularly, the sterically hindered primary amine is an amino acid.

More particularly, the amino acid is a naturally-occurring amino acid.

More particularly, the naturally-occurring amino acid is histidine, phenylalanine, alanine, glutamic acid, aspartic acid, glutamine, leucine, methionine, asparagine, tyrosine, threonine, isoleucine, tryptophan, glycine or valine.

More particularly, the naturally-occurring amino acid is leucine, isoleucine, alanine or methionine.

More particularly, the naturally-occurring amino acid is leucine or methionine.

More particularly, the naturally-occurring amino acid is leucine.

Alternatively, the sterically hindered primary amine is a non-naturally occurring amino acid.

Alternatively, the sterically hindered primary amine has the formula: wherein R₁, R₂ and R₃ are independently selected from: H; —C(O)OH; C₁-C₆ alkyl, optionally substituted by —CO₂H, —CONH₂, or a 5-10 membered aryl or heteroaryl; C₁-C₆ alkoxyalkyl; or C₁-C₆ thioalkoxyalkyl, wherein any of the alkyl or aryl groups above can be singly or multiply substituted with halogen or —NH₂, and provided that no more than two of R₁, R₂ and R₃ are H. In a further embodiment, no more than one of R₁, R₂ and R₃ is H.

More particularly, the sterically hindered primary amine has the formula:

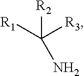

wherein R₁, R₂ and R₃ are independently selected from: H; —C(O)OH; C₁-C₆ alkyl; C₁-C₆ alkoxyalkyl; or C₁-C₆ thioalkoxyalkyl, wherein any of the alkyl or aryl groups above can be singly or multiply substituted with halogen or —NH₂, and provided that no more than two of R₁, R₂ and R₃ are H. In a further embodiment, no more than one of R₁, R₂ and R₃ is H.

More particularly, the sterically hindered primary amine has the formula:

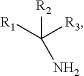

wherein R₁, R₂ and R₃ are independently selected from: H; —C(O)OH; C₁-C₆ alkyl; C₁-C₆ alkoxyalkyl; or C₁-C₆ thioalkoxyalkyl, and provided that no more than two of R₁, R₂ and R₃ are H. In a further embodiment, no more than one of R₁, R₂ and R₃ is H.

In one aspect, the sterically hindered primary amine is cyclohexylamine or 2-methylbutylamine.

In one aspect, the sterically hindered primary amine is chitosan.

In any of these aspects or embodiments, the GC-C receptor agonist polypeptide may be linaclotide.

In another aspect, the GC-C receptor agonist polypeptide formulation used in the method comprises a cation selected from Mg²⁺, Ca²⁺, Zn²⁺, Mn²⁺, K⁺, Na⁺ and Al³⁺ and a sterically hindered primary amine.

More particularly, the cation Mg²⁺, Ca²⁺, Zn²⁺, Mn²⁺, K⁺, Na⁺ or Al³⁺ is provided as magnesium acetate, magnesium chloride, magnesium phosphate, magnesium sulfate, calcium acetate, calcium chloride, calcium phosphate, calcium sulfate, zinc acetate, zinc chloride, zinc phosphate, zinc sulfate, manganese acetate, manganese chloride, manganese phosphate, manganese sulfate, potassium acetate, potassium chloride, potassium phosphate, potassium sulfate, sodium acetate, sodium chloride, sodium phosphate, sodium sulfate, aluminum acetate, aluminum chloride, aluminum phosphate or aluminum sulfate.

More particularly, the cation Mg²⁺, Ca²⁺, Zn²⁺, Mn²⁺, K⁺, Na⁺ or Al³⁺ is provided as magnesium chloride, calcium chloride, calcium phosphate, calcium sulfate, zinc acetate, manganese chloride, potassium chloride, sodium chloride or aluminum chloride.

More particularly, the cation is selected from Mg²⁺, Ca²⁺ and Zn²⁺ and a sterically hindered primary amine.

More particularly, the Mg²⁺, Ca²⁺ or Zn²⁺ is provided as magnesium chloride, calcium chloride or zinc acetate.

More particularly, the cation is Ca²⁺.

More particularly, the Ca²⁺ is provided as calcium chloride.

More particularly, the sterically hindered primary amine is an amino acid.

More particularly, the amino acid is a naturally-occurring amino acid.

More particularly, the naturally-occurring amino acid is histidine, phenylalanine, alanine, glutamic acid, aspartic acid, glutamine, leucine, methionine, asparagine, tyrosine, threonine, isoleucine, tryptophan, glycine or valine.

More particularly, the naturally-occurring amino acid is leucine, isoleucine, alanine or methionine.

More particularly, the naturally-occurring amino acid is leucine or methionine.

More particularly, the naturally-occurring amino acid is leucine.

Alternatively, the sterically hindered primary amine is a non-naturally occurring amino acid.

More particularly, the non-naturally occurring amino acid is 1-aminocyclohexane carboxylic acid.

Alternatively, the sterically hindered primary amine has the formula: wherein R₁, R₂ and R₃ are independently selected from: H; —C(O)OH; C₁-C₆ alkyl, optionally substituted by —CO₂H, —CONH₂, or a 5-10 membered aryl or heteroaryl; C₁-C₆ alkoxyalkyl; or C₁-C₆ thioalkoxyalkyl, wherein any of the alkyl or aryl groups above can be singly or multiply substituted with halogen or —NH₂, and provided that no more than two of R₁, R₂ and R₃ are H. In a further embodiment, no more than one of R₁, R₂ and R₃ is H.

More particularly, the sterically hindered primary amine has the formula:

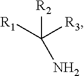

wherein R₁, R₂ and R₃ are independently selected from: H; —C(O)OH; C₁-C₆ alkyl; C₁-C₆ alkoxyalkyl; or C₁-C₆ thioalkoxyalkyl, wherein any of the alkyl or aryl groups above can be singly or multiply substituted with halogen or —NH₂, and provided that no more than two of R₁, R₂ and R₃ are H. In a further embodiment, no more than one of R₁, R₂ and R₃ is H.

More particularly, the sterically hindered primary amine has the formula:

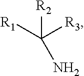

wherein R₁, R₂ and R₃ are independently selected from: H; —C(O)OH; C₁-C₆ alkyl; C₁-C₆ alkoxyalkyl; or C₁-C₆ thioalkoxyalkyl, and provided that no more than two of R₁, R₂ and R₃ are H. In a further embodiment, no more than one of R₁, R₂ and R₃ is H.

In one aspect, the sterically hindered primary amine is cyclohexylamine or 2-methylbutylamine.

In one aspect, the sterically hindered primary amine is chitosan.

In any of these aspects or embodiments, the GC-C receptor agonist polypeptide may be linaclotide.

In these aspects, the GC-C receptor agonist polypeptide formulation additionally comprises a pharmaceutically acceptable binder. In particular, the pharmaceutically acceptable binder is selected from polyvinyl alcohol, polyvinylpyrrolidone (povidone), a starch, maltodextrin or a cellulose ether. More particularly, the pharmaceutically acceptable binder is a cellulose ether which may be selected from: methylcellulose, ethylcellulose, carboxymethylcellulose, hydroxyethyl cellulose, hydroxyethyl methylcellulose, hydroxypropyl cellulose and hydroxypropyl methylcellulose.

In these aspects, the GC-C receptor agonist polypeptide formulation additionally comprises a pharmaceutically acceptable glidant, lubricant or additive that acts as both a glidant and lubricant.

In these aspects, the GC-C receptor agonist polypeptide formulation additionally comprises an antioxidant. In particular, the antioxidant is BHA, vitamin E or propyl gallate.

In these aspects, the GC-C receptor agonist polypeptide formulation additionally comprises a pharmaceutically acceptable filler. In particular, the pharmaceutically acceptable filler is cellulose, isomalt, mannitol or dibasic calcium phosphate.

More particularly, the cellulose used in the filler is selected from microfine cellulose and microcrystalline cellulose. More particularly, the pharmaceutically acceptable filler comprises particles having an average diameter between 150 μm and 1000 μm.

In another aspect, when the sterically hindered primary amine is leucine and the cation is Ca²⁺.

In another aspect, the molar ratio of Ca²⁺ to leucine is at least 1:1.

More particularly, the molar ratio of Ca²⁺ to leucine is at least 1.5:1.

More particularly, the molar ratio of Ca²⁺ to leucine is at least 2:1.

In this aspect, the sterically hindered amine is leucine and the molar ratio of leucine to GC-C receptor agonist polypeptide is at least 10:1.

More particularly, the molar ratio of leucine to GC-C receptor agonist polypeptide is at least 20:1.

More particularly, the molar ratio of leucine to GC-C receptor agonist polypeptide in the pharmaceutical formulation is at least 30:1.

In the GC-C receptor agonist polypeptide formulations comprising a filler, the weight ratio of GC-C receptor agonist polypeptide to pharmaceutically acceptable filler is between 1:25 and 1:2,500.

More particularly, the weight ratio of GC-C receptor agonist polypeptide to pharmaceutically acceptable filler is between 1:100 and 1:2000.

More particularly, the weight ratio of GC-C receptor agonist polypeptide to pharmaceutically acceptable filler is between 1:100 and 1:1000.

In the GC-C receptor agonist polypeptide formulations, comprising a cation and a sterically hindered primary amine, the molar ratio of cation:sterically hindered primary amine:GC-C receptor agonist polypeptide is 40-100:20-50:1.

More particularly, when the cation is Ca²⁺ and the sterically hindered primary amine is leucine, the molar ratio of Ca²⁺:leucine:GC-C receptor agonist polypeptide is 100:30:1, 80:40:1, 80:30:1, 80:20:1, 60:30:1, 60:20:1, 50:30:1, 50:20:1, 40:20:1, 20:20:1, 10:10:1, 10:5:1, 5:10:1 or 5:5:1.

More particularly, the molar ratio of Ca²⁺:leucine:GC-C receptor agonist polypeptide is 60:30:1.

More particularly, the cation s provided as CaCl₂.

In any of these aspects or embodiments, the GC-C receptor agonist polypeptide may be linaclotide.

In another aspect, the GC-C receptor antagonist formulation is in the form of a capsule or tablet.

In particular, each capsule or tablet comprises 50 μg to 1 mg GC-C receptor agonist polypeptide. In some embodiments, each capsule or table t comprises 50 μg to 1 mg linaclotide.

More particularly, each capsule or tablet comprises 100 μg, 133 μg, 150 μg, 200 μg, 266 μg, 300 μg, 400 μg, 500 μg or 600 μg GC-C receptor agonist polypeptide. In some embodiments, each capsule or tablet comprises 100 μg, 133 μg, 150 μg, 200 μg, 266 μg, 300 μg, 400 μg, 500 μg or 600 μg linaclotide.

More particularly, each tablet or capsule comprises:

(a) Linaclotide;

(b) CaCl₂.2H₂O;

(c) L-Leucine; and

(d) Hydroxypropyl Methylcellulose

wherein linaclotide is present in the pharmaceutical composition in an amount between 100 μg to 600 μg and the molar ratio of Ca²⁺:leucine:linaclotide is between 5-100:5-50:1.

More particularly, linaclotide is present in the tablet or capsule in an amount of 133 or 266 μg.

More particularly, CaCl₂ is present in the tablet or capsule in an amount of 1541 μg.

More particularly, leucine is present in the tablet or capsule in an amount of 687 μg.

More particularly, hydroxypropyl methylcellulose is present in the tablet or capsule in an amount of 700 μg.

In another embodiment, the invention provides a method of decreasing the pharmacodynamic effect of a GC-C receptor agonist polypeptide which is administered to a subject in need of such treatment, comprising administering the GC-C receptor agonist polypeptide to the subject a sufficient time period after an ingestion of food

In one aspect of this embodiment, the GC-C receptor agonist polypeptide formulation is administered to the subject at least 6 hours after the ingestion of food.

In another aspect of this embodiment, the GC-C receptor agonist polypeptide formulation is administered to the subject at least 8 hours after the ingestion of food.

In another aspect of this embodiment, the GC-C receptor agonist polypeptide formulation is administered to the subject at least 10 hours after the ingestion of food.

In another aspect of this embodiment, the GC-C receptor agonist polypeptide formulation is administered to the subject at least 15 minutes before the ingestion of more food.

In another aspect of this embodiment, the GC-C receptor agonist polypeptide formulation is administered to the subject at least 30 minutes before the ingestion of more food.

In another aspect of this embodiment, the GC-C receptor agonist polypeptide formulation is administered to the subject at least 1 hour before the ingestion of more food.

In another aspect of this embodiment, the GC-C receptor agonist polypeptide formulation is administered to the subject at least 2 hours before the ingestion of more food.

In these aspects, the GC-C receptor agonist polypeptide may be linaclotide.

In another aspect of this embodiment, the subject in need of such treatment is suffering from irritable bowel syndrome (IBS). More particularly the disorder is IBS, which is constipation-predominant IBS (IBS-c) or alternating IBS (IBS-a). More particularly, the disorder is IBS-c. In some embodiments, the GC-C receptor agonist polypeptide is linaclotide.

In another aspect of this embodiment, the subject in need of such treatment is suffering from constipation. More particularly, the disorder is constipation which is chronic constipation, idiopathic constipation, post-operative ileus, or constipation caused by opiate use. In some embodiments, the GC-C receptor agonist polypeptide is linaclotide.

In these aspects, the GC-C receptor agonist polypeptide is administered as a formulation comprising the GC-C receptor agonist polypeptide and a pharmaceutically acceptable excipient.

In another embodiment, the invention provides a method of increasing the pharmacodynamic effect of a GC-C receptor agonist polypeptide which is administered to a subject in need of such treatment, comprising administering the GC-C receptor agonist polypeptide to the subject with the ingestion of food or within two hours after the ingestion of food.

In one aspect of this embodiment, the GC-C receptor agonist polypeptide is administered with the ingestion of food.

In another aspect of this embodiment, the GC-C receptor agonist polypeptide is administered with a meal.

In another aspect of this embodiment, the GC-C receptor agonist polypeptide is administered within two hours after the ingestion of food.

In another aspect of this embodiment, the GC-C receptor agonist polypeptide is administered within one hour after the ingestion of food.

In another aspect of this embodiment, the GC-C receptor agonist polypeptide is administered within 30 minutes after the ingestion of food.

In another aspect of this embodiment, the GC-C receptor agonist polypeptide is administered within 15 minutes after the ingestion of food.

In another aspect of this embodiment, the GC-C receptor agonist polypeptide is linaclotide.

In another aspect of this embodiment, the subject in need of such treatment is suffering from irritable bowel syndrome (IBS). More particularly the disorder is IBS, which is constipation-predominant IBS (IBS-c) or alternating IBS (IBS-a). More particularly, the disorder is IBS-c.

In another aspect of this embodiment, the subject in need of such treatment is suffering from constipation. More particularly, the disorder is constipation which is chronic constipation, idiopathic constipation, post-operative ileus, or constipation caused by opiate use.

In another aspect of this embodiment, the GC-C receptor agonist polypeptide is administered as a formulation comprising the GC-C receptor agonist polypeptide and a pharmaceutically acceptable excipient.

In another aspect of this embodiment, the formulation further comprises one or more agents selected from a cation selected from Mg²⁺, Ca²⁺, Zn²⁺, Mn²⁺, K⁺, Na⁺ and Al³⁺ or a sterically hindered primary amine.

In particular, the Mg²⁺, Ca²⁺, Zn²⁺, Mn²⁺, K⁺, Na⁺ or Al³⁺ is provided as magnesium acetate, magnesium chloride, magnesium phosphate, magnesium sulfate, calcium acetate, calcium chloride, calcium phosphate, calcium sulfate, zinc acetate, zinc chloride, zinc phosphate, zinc sulfate, manganese acetate, manganese chloride, manganese phosphate, manganese sulfate, potassium acetate, potassium chloride, potassium phosphate, potassium sulfate, sodium acetate, sodium chloride, sodium phosphate, sodium sulfate, aluminum acetate, aluminum chloride, aluminum phosphate or aluminum sulfate.

More particularly, the Mg²⁺, Ca²⁺, Zn²⁺, Mn²⁺, K⁺, Na⁺ or Al³⁺ is provided as magnesium chloride, calcium chloride, calcium phosphate, calcium sulfate, zinc acetate, manganese chloride, potassium chloride, sodium chloride or aluminum chloride.

More particularly, the cation is Mg²⁺, Ca²⁺, Zn²⁺.

More particularly, the Mg²⁺, Ca²⁺ or Zn²⁺ is provided as magnesium chloride, calcium chloride or zinc acetate.

More particularly, the agent is Ca²⁺.

More particularly, the Ca²⁺ is provided as calcium chloride.

Alternatively, the agent is a sterically hindered primary amine.

More particularly, the sterically hindered primary amine is an amino acid.

More particularly, the amino acid is a naturally-occurring amino acid.

More particularly, the naturally-occurring amino acid is histidine, phenylalanine, alanine, glutamic acid, aspartic acid, glutamine, leucine, methionine, asparagine, tyrosine, threonine, isoleucine, tryptophan, glycine or valine.

More particularly, the naturally-occurring amino acid is leucine, isoleucine, alanine or methionine.

More particularly, the naturally-occurring amino acid is leucine or methionine.

More particularly, the naturally-occurring amino acid is leucine.

Alternatively, the sterically hindered primary amine is a non-naturally occurring amino acid.

More particularly, the non-naturally occurring amino acid is 1-aminocyclohexane carboxylic acid.

Alternatively, the sterically hindered primary amine has the formula: wherein R₁, R₂ and R₃ are independently selected from: H; —C(O)OH; C₁-C₆ alkyl, optionally substituted by —CO₂H, —CONH₂, or a 5-10 membered aryl or heteroaryl; C₁-C₆ alkoxyalkyl; or C₁-C₆ thioalkoxyalkyl, wherein any of the alkyl or aryl groups above can be singly or multiply substituted with halogen or —NH₂, and provided that no more than two of R₁, R₂ and R₃ are H. In a further embodiment, no more than one of R₁, R₂ and R₃ is H.

More particularly, the sterically hindered primary amine has the formula:

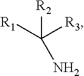

wherein R₁, R₂ and R₃ are independently selected from: H; —C(O)OH; C₁-C₆ alkyl; C₁-C₆ alkoxyalkyl; or C₁-C₆ thioalkoxyalkyl, wherein any of the alkyl or aryl groups above can be singly or multiply substituted with halogen or —NH₂, and provided that no more than two of R₁, R₂ and R₃ are H. In a further embodiment, no more than one of R₁, R₂ and R₃ is H.

More particularly, the sterically hindered primary amine has the formula:

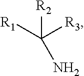

wherein R₁, R₂ and R₃ are independently selected from: H; —C(O)OH; C₁-C₆ alkyl; C₁-C₆ alkoxyalkyl; or C₁-C₆ thioalkoxyalkyl, and provided that no more than two of R₁, R₂ and R₃ are H. In a further embodiment, no more than one of R₁, R₂ and R₃ is H.

In some embodiments, the sterically hindered primary amine is cyclohexylamine or 2-methylbutylamine.

In some embodiments, the sterically hindered primary amine is chitosan.

In some embodiments, the GC-C receptor agonist polypeptide is linaclotide.

In another aspect, the GC-C receptor agonist polypeptide formulation used in the method comprises a cation selected from Mg²⁺, Ca²⁺, Zn²⁺, Mn²⁺, K⁺, Na⁺ and Al³⁺ and a sterically hindered primary amine.

More particularly, the Mg²⁺, Ca²⁺, Zn²⁺, Mn²⁺, K⁺, Na⁺ or Al³⁺ is provided as magnesium acetate, magnesium chloride, magnesium phosphate, magnesium sulfate, calcium acetate, calcium chloride, calcium phosphate, calcium sulfate, zinc acetate, zinc chloride, zinc phosphate, zinc sulfate, manganese acetate, manganese chloride, manganese phosphate, manganese sulfate, potassium acetate, potassium chloride, potassium phosphate, potassium sulfate, sodium acetate, sodium chloride, sodium phosphate, sodium sulfate, aluminum acetate, aluminum chloride, aluminum phosphate or aluminum sulfate.

More particularly, the Mg²⁺, Ca²⁺, Zn²⁺, Mn²⁺, K⁺, Na⁺ or Al³⁺ is provided as magnesium chloride, calcium chloride, calcium phosphate, calcium sulfate, zinc acetate, manganese chloride, potassium chloride, sodium chloride or aluminum chloride.

More particularly, the cation is Mg²⁺, Ca²⁺ or Zn²⁺.

More particularly, the Mg²⁺, Ca²⁺ or Zn²⁺ is provided as magnesium chloride, calcium chloride or zinc acetate.

More particularly, the cation is Ca²⁺.

More particularly, the Ca²⁺ is provided as calcium chloride.

More particularly, the sterically hindered primary amine is an amino acid.

More particularly, the amino acid is a naturally-occurring amino acid.

More particularly, the naturally-occurring amino acid is histidine, phenylalanine, alanine, glutamic acid, aspartic acid, glutamine, leucine, methionine, asparagine, tyrosine, threonine, isoleucine, tryptophan, glycine or valine.

More particularly, the naturally-occurring amino acid is leucine, isoleucine, alanine or methionine.

More particularly, the naturally-occurring amino acid is leucine or methionine.

More particularly, the naturally-occurring amino acid is leucine.

Alternatively, the sterically hindered primary amine is a non-naturally occurring amino acid.

More particularly, the non-naturally occurring amino acid is 1-aminocyclohexane carboxylic acid.

Alternatively, the sterically hindered primary amine has the formula: wherein R₁, R₂ and R₃ are independently selected from: II; —C(O)OH; C₁-C₆ alkyl, optionally substituted by —CO₂H, —CONH₂, or a 5-10 membered aryl or heteroaryl; C₁-C₆ alkoxyalkyl; or C₁-C₆ thioalkoxyalkyl, wherein any of the alkyl or aryl groups above can be singly or multiply substituted with halogen or —NH₂, and provided that no more than two of R₁, R₂ and R₃ are H. In a further embodiment, no more than one of R₁, R₂ and R₃ is H.

More particularly, the sterically hindered primary amine has the formula:

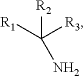

wherein R₁, R₂ and R₃ are independently selected from: H; —C(O)OH; C₁-C₆ alkyl; C₁-C₆ alkoxyalkyl; or C₁-C₆ thioalkoxyalkyl, wherein any of the alkyl or aryl groups above can be singly or multiply substituted with halogen or —NH₂, and provided that no more than two of R₁, R₂ and R₃ are H. In a further embodiment, no more than one of R₁, R₂ and R₃ is H.

More particularly, the sterically hindered primary amine has the formula:

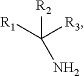

wherein R₁, R₂ and R₃ are independently selected from: H; —C(O)OH: C₁-C₆ alkyl; C₁-C₆ alkoxyalkyl; or C₁-C₆ thioalkoxyalkyl, and provided that no more than two of R₁, R₂ and R₃ are H. In a further embodiment, no more than one of R₁, R₂ and R₃ is H.

In some embodiments, the sterically hindered primary amine is cyclohexylamine or 2-methylbutylamine.

In some embodiments, the sterically hindered primary amine is chitosan.

In some embodiments, the GC-C receptor agonist polypeptide is linaclotide.

In these aspects, the GC-C receptor agonist polypeptide formulation additionally comprises a pharmaceutically acceptable binder. In particular, the pharmaceutically acceptable binder is selected from polyvinyl alcohol, polyvinylpyrrolidone (povidone), a starch, maltodextrin or a cellulose ether. More particularly, the pharmaceutically acceptable binder is a cellulose ether which may be selected from: methylcellulose, ethylcellulose, carboxymethylcellulose, hydroxyethyl cellulose, hydroxyethyl methylcellulose, hydroxypropyl cellulose and hydroxypropyl methylcellulose.

In these aspects, the GC-C receptor agonist polypeptide formulation additionally comprises a pharmaceutically acceptable glidant, lubricant or additive that acts as both a glidant and lubricant.

In these aspects, the GC-C receptor agonist polypeptide formulation additionally comprises an antioxidant. In particular, the antioxidant is BHA, vitamin E or propyl gallate.

In these aspects, the GC-C receptor agonist polypeptide formulation additionally comprises a pharmaceutically acceptable filler. In particular, the pharmaceutically acceptable filler is cellulose, isomalt, mannitol or dibasic calcium phosphate.

More particularly, the cellulose used in the filler is selected from microtine cellulose and microcrystalline cellulose. More particularly, the pharmaceutically acceptable filler comprises particles having an average diameter between 150 μm and 1000 μm.

In another aspect, when the sterically hindered primary amine is leucine and the cation is Ca²⁺.

In another aspect, the molar ratio of Ca²⁺ to leucine is at least 1:1.

More particularly, the molar ratio of Ca²⁺ to leucine is at least 1.5:1.

More particularly, the molar ratio of Ca²⁺ to leucine is at least 2:1.

In this aspect, the sterically hindered amine is leucine and the molar ratio of leucine to GC-C receptor agonist polypeptide is at least 10:1.

More particularly, the molar ratio of leucine to GC-C receptor agonist polypeptide is at least 20:1.

More particularly, the molar ratio of leucine to GC-C receptor agonist polypeptide in the pharmaceutical formulation is at least 30:1.

In the GC-C receptor agonist polypeptide formulations comprising a filler, the weight ratio of GC-C receptor agonist polypeptide to pharmaceutically acceptable filler is between 1:25 and 1:2,500.

More particularly, the weight ratio of GC-C receptor agonist polypeptide to pharmaceutically acceptable filler is between 1:100 and 1:2000.

More particularly, the weight ratio of GC-C receptor agonist polypeptide to pharmaceutically acceptable filler is between 1:100 and 1:1000.

In the GC-C receptor agonist polypeptide formulations, comprising a cation and a sterically hindered primary amine, the molar ratio of cation:sterically hindered primary amine:GC-C receptor agonist polypeptide is 40-100:20-50:1.

More particularly, when the cation is Ca²⁺ and the sterically hindered primary amine is leucine, the molar ratio of Ca²⁺:leucine:GC-C receptor agonist polypeptide is 100:30:1, 80:40:1, 80:30:1, 80:20:1, 60:30:1, 60:20:1, 50:30:1, 50:20:1, 40:20:1, 20:20:1, 10:10:1, 10:5:1, 5:10:1 or 5:5:1.

More particularly, the molar ratio of Ca²⁺:leucine:GC-C receptor agonist polypeptide is 60:30:1.

More particularly, the cation is provided as CaCl₂.

In some embodiments, the GC-C receptor agonist polypeptide is linaclotide.

In another aspect of the invention method, the GC-C receptor antagonist formulation is in the form of a capsule or tablet.

In particular, each capsule or tablet comprises 50 μg to 1 mg GC-C receptor agonist polypeptide. In some embodiments, each capsule or tablet comprises 50 μg to 1 mg linaclotide.

More particularly, each capsule or tablet comprises 100 μg, 150 μg, 200 μg, 300 μg, 400 μg, 500 μg or 600 μg GC-C receptor agonist polypeptide. In some embodiments, each capsule or tablet comprises 100 μg, 133 μg, 150 μg, 200 μg, 266 μg, 300 μg, 400 μg, 500 μg or 600 μg linaclotide.

More particularly, each tablet or capsule comprises:

(a) Linaclotide;

(b) CaCl₂.2H₂O;

(c) L-Leucine; and

(d) Hydroxypropyl Methylcellulose

wherein linaclotide is present in the pharmaceutical composition in an amount between 100 μg to 600 μg and the molar ratio of Ca²⁺:leucine:linaclotide is between 5-100:5-50:1.

More particularly, linaclotide is present in the tablet or capsule in an amount of 133 or 266 μg.

More particularly, CaCl₂ is present in the tablet or capsule in an amount of 1541 μg.

More particularly, leucine is present in the tablet or capsule in an amount of 687 μg.

More particularly, hydroxypropyl methylcellulose is present in the tablet or capsule in an amount of 700 μg.

In another embodiment, the invention provides a method of treating irritable bowel syndrome (e.g., IBS-c) or constipation (e.g., chronic constipation) in a subject in need of such treatment, comprising: administering a GC-C receptor agonist polypeptide to the subject before the ingestion of food.

In one aspect of this embodiment, the GC-C receptor agonist polypeptide formulation is administered to the subject at least 6 hours after the ingestion of food.

In another aspect of this embodiment, the GC-C receptor agonist polypeptide formulation is administered to the subject at least 8 hours after the ingestion of food.

In another aspect of this embodiment, the GC-C receptor agonist polypeptide formulation is administered to the subject at least 10 hours after the ingestion of food.

In another aspect of this embodiment, the GC-C receptor agonist polypeptide formulation is administered to the subject at least 15 minutes before the ingestion of more food.

In another aspect of this embodiment, the GC-C receptor agonist polypeptide formulation is administered to the subject at least 30 minutes before the ingestion of more food.

In another aspect of this embodiment, the GC-C receptor agonist polypeptide formulation is administered to the subject at least 1 hour before the ingestion of more food.

In another aspect of this embodiment, the GC-C receptor agonist polypeptide formulation is administered to the subject at least 2 hours before the ingestion of more food.

In these aspects, the GC-C receptor agonist polypeptide may be linaclotide.

In another aspect of this embodiment, the subject in need of such treatment is suffering from irritable bowel syndrome (IBS). More particularly the disorder is IBS, which is constipation-predominant IBS (IBS-c) or alternating IBS (IBS-a). More particularly, the disorder is IBS-c.

In another aspect of this embodiment, the subject in need of such treatment is suffering from constipation. More particularly, the disorder is constipation which is chronic constipation, idiopathic constipation, post-operative ileus, or constipation caused by opiate use.

In these aspects, the GC-C receptor agonist polypeptide is administered as a formulation comprising the GC-C receptor agonist polypeptide and a pharmaceutically acceptable excipient.

In another embodiment, the invention provides a method of increasing the pharmacodynamic effect of a GC-C receptor agonist polypeptide which is administered to a subject in need of such treatment, comprising administering the GC-C receptor agonist polypeptide to the subject with the ingestion of food or within two hours after the ingestion of food.

In one aspect of this embodiment, the GC-C receptor agonist polypeptide is administered with the ingestion of food.

In another aspect of this embodiment, the GC-C receptor agonist polypeptide is administered with a meal.

In another aspect of this embodiment, the GC-C receptor agonist polypeptide is administered within two hours after the ingestion of food.

In another aspect of this embodiment, the GC-C receptor agonist polypeptide is administered within one hour after the ingestion of food.

In another aspect of this embodiment, the GC-C receptor agonist polypeptide is administered within 30 minutes after the ingestion of food.

In another aspect of this embodiment, the GC-C receptor agonist polypeptide is administered within 15 minutes after the ingestion of food.

In another aspect of this embodiment, the GC-C receptor agonist polypeptide is linaclotide.

In another aspect of this embodiment, the subject in need of such treatment is suffering from irritable bowel syndrome (IBS). More particularly the disorder is IBS, which is constipation-predominant IBS (IBS-c) or alternating IBS (IBS-a). More particularly, the disorder is IBS-c.

In another aspect of this embodiment, the subject in need of such treatment is suffering from constipation. More particularly, the disorder is constipation which is chronic constipation, idiopathic constipation, post-operative ileus, or constipation caused by opiate use.

In another aspect of this embodiment, the GC-C receptor agonist polypeptide is administered as a formulation comprising the GC-C receptor agonist polypeptide and a pharmaceutically acceptable excipient.

In another aspect of this embodiment, the formulation further comprises one or more agents selected from a cation selected from Mg²⁺, Ca²⁺, Zn²⁺, Mn²⁺, K⁺, Na⁺ and Al³⁺ or a sterically hindered primary amine.

In particular, the Mg²⁺, Ca²⁺, Zn²⁺, Mn²⁺, K⁺, Na⁺ or Al³⁺ is provided as magnesium acetate, magnesium chloride, magnesium phosphate, magnesium sulfate, calcium acetate, calcium chloride, calcium phosphate, calcium sulfate, zinc acetate, zinc chloride, zinc phosphate, zinc sulfate, manganese acetate, manganese chloride, manganese phosphate, manganese sulfate, potassium acetate, potassium chloride, potassium phosphate, potassium sulfate, sodium acetate, sodium chloride, sodium phosphate, sodium sulfate, aluminum acetate, aluminum chloride, aluminum phosphate or aluminum sulfate.

More particularly, the Mg²⁺, Ca²⁺, Zn²⁺, Mn²⁺, K⁺, Na⁺ or Al³⁺ is provided as magnesium chloride, calcium chloride, calcium phosphate, calcium sulfate, zinc acetate, manganese chloride, potassium chloride, sodium chloride or aluminum chloride.

More particularly, the cation is Mg²⁺, Ca²⁺, Zn²⁺.

More particularly, the Mg²⁺, Ca²⁺ or Zn²⁺ is provided as magnesium chloride, calcium chloride or zinc acetate.

More particularly, the agent is Ca²⁺.

More particularly, the Ca²⁺ is provided as calcium chloride.

Alternatively, the agent is a sterically hindered primary amine.

More particularly, the sterically hindered primary amine is an amino acid.

More particularly, the amino acid is a naturally-occurring amino acid.

More particularly, the naturally-occurring amino acid is histidine, phenylalanine, alanine, glutamic acid, aspartic acid, glutamine, leucine, methionine, asparagine, tyrosine, threonine, isoleucine, tryptophan, glycine or valine.

More particularly, the naturally-occurring amino acid is leucine, isoleucine, alanine or methionine.

More particularly, the naturally-occurring amino acid is leucine or methionine.

More particularly, the naturally-occurring amino acid is leucine.

Alternatively, the sterically hindered primary amine is a non-naturally occurring amino acid.

More particularly, the non-naturally occurring amino acid is 1-aminocyclohexane carboxylic acid.

Alternatively, the sterically hindered primary amine has the formula: wherein R₁, R₂ and R₃ are independently selected from: H; —C(O)OH; C₁-C₆ alkyl, optionally substituted by —CO₂H, —CONH₂, or a 5-10 membered aryl or heteroaryl; C₁-C₆ alkoxyalkyl; or C₁-C₆ thioalkoxyalkyl, wherein any of the alkyl or aryl groups above can be singly or multiply substituted with halogen or —NH₂, and provided that no more than two of R₁, R₂ and R₃ are H. In a further embodiment, no more than one of R₁, R₂ and R₃ is H.

More particularly, the sterically hindered primary amine has the formula:

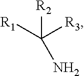

wherein R₁, R₂ and R₃ are independently selected from: H; —C(O)OH; C₁-C₆ alkyl; C₁-C₆ alkoxyalkyl; or C₁-C₆ thioalkoxyalkyl, wherein any of the alkyl or aryl groups above can be singly or multiply substituted with halogen or —NH₂, and provided that no more than two of R₁, R₂ and R₃ are H. In a further embodiment, no more than one of R₁, R₂ and R₃ is H.

More particularly, the sterically hindered primary amine has the formula:

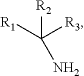

wherein R₁, R₂ and R₃ are independently selected from: H; —C(O)OH; C₁-C₆ alkyl; C₁-C₆ alkoxyalkyl; or C₁-C₆ thioalkoxyalkyl, and provided that no more than two of R₁, R₂ and R₃ are H. In a further embodiment, no more than one of R₁, R₂ and R₃ is H.

In some embodiments, the sterically hindered primary amine is cyclohexylamine or 2-methylbutylamine.

In some embodiments, the sterically hindered primary amine is chitosan.

In some embodiments, the GC-C receptor agonist polypeptide is linaclotide.

In another aspect, the GC-C receptor agonist polypeptide formulation used in the method comprises a cation selected from Mg²⁺, Ca²⁺, Zn²⁺, Mn²⁺, K⁺, Na⁺ and Al³⁺ and a sterically hindered primary amine.

More particularly, the Mg²⁺, Ca²⁺, Zn²⁺, Mn²⁺, K⁺, Na⁺ or Al³⁺ is provided as magnesium acetate, magnesium chloride, magnesium phosphate, magnesium sulfate, calcium acetate, calcium chloride, calcium phosphate, calcium sulfate, zinc acetate, zinc chloride, zinc phosphate, zinc sulfate, manganese acetate, manganese chloride, manganese phosphate, manganese sulfate, potassium acetate, potassium chloride, potassium phosphate, potassium sulfate, sodium acetate, sodium chloride, sodium phosphate, sodium sulfate, aluminum acetate, aluminum chloride, aluminum phosphate or aluminum sulfate.

More particularly, the Mg²⁺, Ca²⁺, Zn²⁺, Mn²⁺, K⁺, Na⁺ or Al³⁺ is provided as magnesium chloride, calcium chloride, calcium phosphate, calcium sulfate, zinc acetate, manganese chloride, potassium chloride, sodium chloride or aluminum chloride.

More particularly, the cation is Mg²⁺, Ca²⁺ or Zn²⁺.

More particularly, the Mg²⁺, Ca²⁺ or Zn²⁺ is provided as magnesium chloride, calcium chloride or zinc acetate.

More particularly, the cation is Ca²⁺.

More particularly, the Ca²⁺ is provided as calcium chloride.

More particularly, the sterically hindered primary amine is an amino acid.

More particularly, the amino acid is a naturally-occurring amino acid.

More particularly, the naturally-occurring amino acid is histidine, phenylalanine, alanine, glutainic acid, aspartic acid, glutamine, leucine, methionine, asparagine, tyrosine, threonine, isoleucine, tryptophan, glycine or valine.

More particularly, the naturally-occurring amino acid is leucine, isoleucine, alanine or methionine.

More particularly, the naturally-occurring amino acid is leucine or methionine.

More particularly, the naturally-occurring amino acid is leucine.

Alternatively, the sterically hindered primary amine is a non-naturally occurring amino acid.

More particularly, the non-naturally occurring amino acid is 1-aminocyclohexane carboxylic acid.

Alternatively, the sterically hindered primary amine has the formula: wherein R₁, R₂ and R₃ are independently selected from; H; —C(O)OH; C₁-C₆ alkyl, optionally substituted by —CO₂H, —CONH₂, or a 5-10 membered aryl or heteroaryl; C₁-C₆ alkoxyalkyl; or C₁-C₆ thioalkoxyalkyl, wherein any of the alkyl or aryl groups above can be singly or multiply substituted with halogen or —NH₂, and provided that no more than two of R₁, R₂ and R₃ are H. In a further embodiment, no more than one of R₁, R₂ and R₃ is H.

More particularly, the sterically hindered primary amine has the formula:

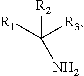

wherein R₁, R₂ and R₃ are independently selected from: H; —C(O)OH; C₁-C₆ alkyl; C₁-C₆ alkoxyalkyl; or C₁-C₆ thioalkoxyalkyl, wherein any of the alkyl or aryl groups above can be singly or multiply substituted with halogen or —NH₂, and provided that no more than two of R₁, R₂ and R₃ are H. In a further embodiment, no more than one of R₁, R₂ and R₃ is H.

More particularly, the sterically hindered primary amine has the formula:

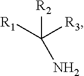

wherein R₁, R₂ and R₃ are independently selected from: H; —C(O)OH; C₁-C₆ alkyl; C₁-C₆ alkoxyalkyl; or C₁-C₆ thioalkoxyalkyl, and provided that no more than two of R₁, R₂ and R₃ are H. In a further embodiment, no more than one of R₁, R₂ and R₃ is H.

More particularly, the sterically hindered primary amine is cyclohexylamine or 2-methylbutylamine.

More particularly, the sterically hindered primary amine is chitosan.

In some embodiments, the GC-C receptor agonist polypeptide is linaclotide.

In these aspects, the GC-C receptor agonist polypeptide formulation additionally comprises a pharmaceutically acceptable binder. In particular, the pharmaceutically acceptable binder is selected from polyvinyl alcohol, polyvinylpyrrolidone (povidone), a starch, maltodextrin or a cellulose ether. More particularly, the pharmaceutically acceptable binder is a cellulose ether which may be selected from: methylcellulose, ethylcellulose, carboxymethylcellulose, hydroxyethyl cellulose, hydroxyethyl methylcellulose, hydroxypropyl cellulose and hydroxypropyl methylcellulose.

In these aspects, the GC-C receptor agonist polypeptide formulation additionally comprises a pharmaceutically acceptable glidant, lubricant or additive that acts as both a glidant and lubricant.

In these aspects, the GC-C receptor agonist polypeptide formulation additionally comprises an antioxidant. In particular, the antioxidant is BHA, vitamin E or propyl gallate.

In these aspects, the GC-C receptor agonist polypeptide formulation additionally comprises a pharmaceutically acceptable filler. In particular, the pharmaceutically acceptable filler is cellulose, isomalt, mannitol or dibasic calcium phosphate.

More particularly, the cellulose used in the filler is selected from microfine cellulose and microcrystalline cellulose. More particularly, the pharmaceutically acceptable filler comprises particles having an average diameter between 150 μm and 1000 μm.

In another aspect, when the sterically hindered primary amine is leucine and the cation is Ca²⁺.

In another aspect, the molar ratio of Ca²⁺ to leucine is at least 1:1.

More particularly, the molar ratio of Ca²⁺ to leucine is at least 1.5:1.

More particularly, the molar ratio of Ca²⁺ to leucine is at least 2:1.

In this aspect, the sterically hindered amine is leucine and the molar ratio of leucine to GC-C receptor agonist polypeptide is at least 10:1.

More particularly, the molar ratio of leucine to GC-C receptor agonist polypeptide is at least 20:1.

More particularly, the molar ratio of leucine to GC-C receptor agonist polypeptide in the pharmaceutical formulation is at least 30:1.

In the GC-C receptor agonist polypeptide formulations comprising a filler, the weight ratio of GC-C receptor agonist polypeptide to pharmaceutically acceptable filler is between 1:25 and 1:2,500.

More particularly, the weight ratio of GC-C receptor agonist polypeptide to pharmaceutically acceptable filler is between 1:100 and 1:2000.

More particularly, the weight ratio of GC-C receptor agonist polypeptide to pharmaceutically acceptable filler is between 1:100 and 1:1000.

In the GC-C receptor agonist polypeptide formulations, comprising a cation and a sterically hindered primary amine, the molar ratio of cation:sterically hindered primary amine:GC-C receptor agonist polypeptide is 40-100:20-50:1.

More particularly, when the cation is Ca²⁺ and the sterically hindered primary amine is leucine, the molar ratio of Ca²⁺:leucine:GC-C receptor agonist polypeptide is 100:30:1, 80:40:1, 80:30:1, 80:20:1, 60:30:1, 60:20:1, 50:30:1, 50:20:1, 40:20:1, 20:20:1, 10:10:1, 10:5:1, 5:10:1 or 5:5:1.

More particularly, the molar ratio of Ca²⁺:leucine:GC-C receptor agonist polypeptide is 60:30:1.

More particularly, the cation is provided as CaCl₂.

In another aspect of the invention method, the GC-C receptor antagonist formulation is in the form of a capsule or tablet.

In particular, each capsule or tablet comprises 50 μg to 1 mg GC-C receptor agonist polypeptide. In some embodiments, each capsule or tablet comprises 50 μg to 1 mg linaclotide.

More particularly, each capsule or tablet comprises 100 μg, 150 μg, 200 μg, 300 μg, 400 μg, 500 μg or 600 μg GC-C receptor agonist polypeptide. In some embodiments, each capsule or tablet comprises 100 μg, 150 μg, 200 μg, 300 μg, 400 μg, 500 μg or 600 μg linaclotide.

More particularly, each tablet or capsule comprises:

(a) Linaclotide;

(b) CaCl₂.2H₂O;

(c) L-Leucine; and

(d) Hydroxypropyl Methylcellulose

wherein linaclotide is present in the pharmaceutical composition in an amount between 100 μg to 600 μg and the molar ratio of Ca²⁺:leucine:linaclotide is between 5-100; 5-50:1.

More particularly, linaclotide is present in the tablet or capsule in an amount of 133 or 266 μg.

More particularly, CaCl₂ is present in the tablet or capsule in an amount of 1541 μg.

More particularly, leucine is present in the tablet or capsule in an amount of 687 μg.

More particularly, hydroxypropyl methylcellulose is present in the tablet or capsule in an amount of 700 μg.

### EXAMPLES

### Linaclotide Food Effect Study

Linaclotide is a 14 amino acid polypeptide that binds to and activates the guanylate cyclase receptor subtype C (GC-C) receptor on the luminal surface of intestinal enterocytes, resulting in an increase in fluid secretion into the lumen of the intestine and acceleration of colonic transit. Linaclotide has also demonstrated mitigating effects on visceral hypersensitivity in animal models. Linaclotide is being developed as an orally administered therapeutic for the treatment of chronic constipation (CC), irritable bowel syndrome with constipation (IBS-C), and other gastrointestinal (GI) disorders.

The randomized, open-label, two-period, two-sequence, crossover trial of oral linaclotide in healthy volunteers tested under fasted or fed conditions summarized herein was undertaken to determine if linaclotide is susceptible to a food effect. The study consisted of 4 stages: an 8-day to 15-day Screening Stage, two 15- to 16-day Crossover Periods (1 and 2), and a 21-day Washout Stage which was to occur between Crossover Periods 1 and 2. The doses tested in this study were 300 ug and a 1-day dose of 3000 ug at the end of Crossover Periods 1 and 2.

### Objectives

The objective of this study was to compare the pharmacodynamic (PD) effect on stool consistency of linaclotide administered under fed and fasting conditions.

### Methodology

The study consisted of 4 stages: an 8-day to 15-day Screening Stage which was to take place at home and determine eligibility, two 15- to 16-day stages (Crossover Periods 1 and 2) which were to take place in a Phase 1 unit, and a 21-day Washout Stage which was to take place at home and occur between Crossover Periods 1 and 2. The individual study Stages are detailed below.

Screening Stage:

The Screening Stage was to start with the signature of the informed consent form (ICF) at Visit 1 and lasted for 8 to 15 days. During this Stage, eligible healthy volunteers were to be instructed to follow a specific diet (similar to the diet subjects received during Crossover Periods 1 and 2) and to keep a paper based daily diary of their bowel habits (the Bowel Habit Diary, BHD). In this diary, subjects were to record their bowel movements (BMs) during a particular day and provide the date, time, and consistency of each BM. They were also to assess the degree of straining associated with each BM and whether each BM was associated with a sense of complete evacuation. Subjects were not allowed to use laxatives, enemas, and suppositories during the Screening Stage. At the end of the Screening Stage, which was the start of Crossover Period 1, subjects were to report to the Phase 1 unit to have their eligibility for randomization determined.

Crossover Periods 1 and 2:

Eligible subjects were entered into the Phase 1 unit and were randomized to 1 of 2 Treatment Sequences. Sequence 1 (Fed-Fasted): During Crossover Period 1, linaclotide was to be administered immediately after a high-fat breakfast (the fed condition); during Crossover Period 2, linaclotide was to be administered after a 10-hour fast (the fasted condition). Sequence 2 (Fasted-Fed): During Crossover Period 1, linaclotide was to be administered under the fasted condition; during Crossover Period 2, linaclotide was to be administered under the fed condition.

Crossover Period 1 lasted for 15 days and Crossover Period 2 started on Day 35, immediately after the 21-day Washout Stage, and lasted for 16 days. Subjects were to record their bowel habits in their BHD and ingest their meals at the time determined by their randomization sequence. At a specified time on each day, the information in the BHD was to be reviewed by the site staff to insure appropriate completion.

During the Pretreatment Phase of each Crossover Period, subjects were not to receive study drug. On Day 8 of Crossover Period 1 and Day 43 of Crossover Period 2, subjects started the Treatment Phase and were to receive 300 ug linaclotide once daily for a total of 7 days. On Day 14 and 15 of Crossover Period 1, blood was to be drawn to assay for linaclotide and its active metabolite. After the study procedures were completed on Day 15, subjects were discharged home with instructions to follow the same outpatient diet that they followed during the Screening Stage and to return to the Phase 1 unit on Day 35 for Crossover Period 2. On Day 50 of Crossover Period 2, subjects were to receive a single oral dose of 3000 ug linaclotide and were discharged from the Phase 1 unit on Day 51. After subjects received the 3000 ug dose, blood was to be drawn on Day 50 and 51 to assay for linaclotide and linaclotide metabolites.

Linaclotide was to be administered at the same time each morning with 240 cc (8 oz) of water; food was to be withheld for approximately 4 hr after dosing, and additional water was to be withheld from 1 hour before linaclotide administration until 1 hour after dosing. Under the fed condition, subjects were required to start and complete a high-fat breakfast during the 30 minutes before linaclotide administration. Under the fasted condition, linaclotide was to be administered following an overnight fast.

All meals and meal times were standardized during Crossover Periods 1 and 2 with the total daily calorie, fat, and fiber intake the same for each diet condition (fed and fasted). The daily quantity of these nutrients was based on the “United States Department of Agriculture (USDA) Dietary Recommendations for Americans.” Each subject received approximately 2000 total calories per day.

Number of Subjects (Planned and Analyzed):

20 subjects were planned so that at least 16 subjects completed both Crossover Periods; 20 subjects were enrolled and 18 completed both Crossover Periods. Subjects withdrawing from the study were not replaced.

Diagnosis and Main Criteria for Inclusion:

Males and females (non pregnant and non breast feeding) aged 18 to 65 years; Body Mass Index score was ≧18.5 and <35 at the Screening Visit; in good health as determined by medical history, physical examination, 12-lead electrocardiogram (ECG), and vital signs; subject had 4 to 14 BMs and a Bristol Stool Form Scale (BSFS) Score for stool consistency of 2 to 5 for each bowel movement during the last 7 days of the 8-day to 15-day Screening Stage.

Test Product, Dose and Mode of Administration:

Linaclotide, 300 ug, oral gelatin capsule. Linaclotide, 3000 ug (5 oral gelatin capsules of 600 ug linaclotide each), single dose.

Duration of Treatment:

The 300 ug dose of linaclotide was to be administered for a total of 14 days. Since this was a crossover study, each subject was to receive two 7-day courses of the 300 ug doses with each course being separated by 28 days (a 21-day Washout Stage and a 7-day Pretreatment Phase). The 3000 ug dose of linaclotide was to be administered as a single oral dose on Day 50 only. Total subject participation lasted for 66 days.

Criteria for Evaluation:

Pharmacodynamic (PD):

The primary endpoint was the change from Pretreatment Phase to Treatment Phase for each Crossover Period in stool consistency on the BSFS scale. For each of the Pretreatment and Treatment Phases, a subject's BSFS Score was the average of the BSFS Scores for each spontaneous bowel movement (SBM) occurring during that week. The secondary endpoints were SBM frequency, complete spontaneous bowel movement (CSBM) frequency, and degree of straining. An additional analysis of the primary and secondary endpoints was completed based on the last 4 days of treatment (Sensitivity Analysis).

Pharmacokinetic (PK):

Blood samples were to be collected at 0 hour (prior to dosing) and 0.5, 1, 2, 3, 4, 6 and 24 hours post dose on Day 14 of Crossover Period 1 and on Day 50 of Crossover Period 2, for determination of the following PK parameters for linaclotide and a metabolite of linaclotide (CCEYCCNPACTGC (SEQ ID NO: 10)) (if systemic levels of linaclotide and or the metabolite are detected): maximum observed plasma concentration (Cₘₐₓ), time to maximum concentration (Tₘₐₓ), area under the plasma concentration time curve (AUC₀₋ₜ), area under the plasma concentration time curve extrapolated to infinity (AUC_{0-∞}), clearance relative to bioavailability (CL/F), apparent volume of distribution (Vd/F), apparent terminal half life (t_{1/2}).

Statistical Methods:

Primary Endpoint Analysis:

The primary endpoint analysis was a 90 percent confidence interval of the difference in the effect of linaclotide administered in a fasting versus fed condition on the change from Pretreatment BSFS Scores. Equivalence margins of ±0.6125 were used such that if the 90 percent confidence interval was contained within the equivalence margins, the study would have demonstrated equivalence between the fasting and fed conditions relative to stool consistency.

Sample Size Determination:

For this cross-over design study, a sample size of 20 randomized subjects provided 80 percent power to reject the non-equivalence null hypothesis based upon the primary endpoint analysis under the following set of assumptions: 16 of the 20 subjects completed the study and were included in the Per-Protocol population, the standard deviation of the within subject change from Pretreatment to Treatment in BSFS Scores was 0.75, and the expected difference between the fed and fasting conditions was 0.

The primary analysis population for the analysis was the Per-protocol Population. All subjects who completed the study with no major protocol violations were included in the Per-protocol Population.

### Summary of Results

For the Pre-protocol Population, the mean subject age for all subjects was 34.6 years. The mean age for the Fed-Fasted Treatment Sequence was higher (37.3 years) when compared with the Fasted-Fed Treatment Sequence (31.9 years). The difference appeared to be driven by the enrollment of a 62-year-old subject into the Fed-Fasted Treatment Sequence. The majority of subjects were male (83.3 percent) and white (72.2 percent). The percentage of male subjects was higher in the Fasted-Fed Treatment Sequence when compared with the Fed-Fasted Treatment Sequence. African American subjects comprised 27.8 percent of the study population; 11.1 percent of subjects reported Hispanic/Latino ethnicity.

PD Results:

The primary endpoint analysis, the 90 percent confidence interval of the difference between linaclotide in the fasted versus fed condition on the change from pretreatment BSFS Scores, did not demonstrate equivalence, as the confidence interval (0.25, 0.98) was not within the equivalence margins of ±0.6125. This result indicates that taking linaclotide in the fed condition results in looser stools (higher BSFS scores) compared with taking it in the fasted condition. The statistically significantly higher number of SBMs and CSBMs in the fed condition versus the fasted condition also indicates that the fed condition enhances the PD effects of linaclotide. Table 6 provides a summary of bowel habits data for the fed and fasted conditions.

TABLE 6
Summary of Bowel Habits Data for the Fed and Fasted Conditions
(Over 7 days of Treatment)

| | Change from Pretreatment | | | |
|---|---|---|---|---|
| | Mean (SEM) | | Difference | |
| | Linaclotide | Linaclotide | Mean and 90% CI | P-Value |
| PD Score | (300 ug) Fed | (300 ug) Fasted | Fed-Fasted Difference | Fed vs Fasted |
| BSFS Score | 2.45 (0.159) | 1.84 (0.177) | 0.61 (0.25, 0.98)^{c} | 0.0092 |
| SBM | 5.94 (1.503) | 1.50 (0.487) | 4.44 (2.22, 6.67) | 0.0031 |
| CSBM | 4.06 (1.181) | 1.00 (0.443) | 3.06 (0.90, 5.21) | 0.0251 |
| Straining Score | −0.16 (0.064) | −0.17 (0.057) | 0.01 (−0.12, 0.14) | 0.9387 |

CI = Confidence interval
^{c}Did not demonstrate equivalence between the fed and fasted conditions because the 90 percent confidence interval was not within the equivalence margins of ±0.6125.

For the last 4 days of treatment (Sensitivity Analysis), the mean Fed-Fasted difference in BSFS Score was lower than it was for the entire 7 days of treatment, but the 90 percent CI (−0.09, 0.86) still did not fall within the equivalence margins (±0.6125) defined for the primary efficacy endpoint. This result indicates that the enhanced PD effects during the fed condition are present during the last 4 days of treatment but are somewhat less than the results over the 7 days of treatment. Table 7 provides a summary of bowel habits data for the fed and fasted conditions over the last 4 days of treatment for BSFS; and SBM and CSBM Frequency.

TABLE 7
Summary of Bowel Habits Data for the Fed and Fasted Conditions
(Last 4 Days of Treatment)

| | Change from Pretreatment | | Difference Mean | |
|---|---|---|---|---|
| | Mean (SEM) | | and 90 percent | P-Value |
| | Linaclotide | Linaclotide | CI Fed-Fasted | Fed vs |
| PD Score | (300 ug) Fed | (300 ug) Fasted | Difference | Fasted |
| BSFS Score | 2.36 (0.230) | 1.97 (0.174) | 0.38 (−0.09, 0.86) | 0.1754 |
| SBM | 2.28 (0.704) | 0.72 (0.419) | 1.56 (0.60, 2.51) | 0.0118 |
| CSBM | 1.00 (0.647) | 0.39 (0.293) | 0.61 (−0.49, 1.71) | 0.3454 |

CI = Confidence interval

PK Results:

Plasma from all subjects who were administered 300 ug of linaclotide for 7 days of treatment showed no quantifiable levels of linaclotide (limit of detection=0.2 ng/ml) or linaclotide metabolite (limit of detection=2.0 ng/ml). The resulting bioanalysis of the 18 subjects who received 10 times this dose (3000 ug), yielded only 2 individuals (subject numbers 001012 and 001019, both in the fasted condition) with detectable levels of linaclotide with Cₘₐₓ (Tₘₐₓ) being 0.735 ng/ml (2 h) and 0.212 ng/ml (0.5 h), respectively in the plasma. Subject 001019 had only a single sample with a measurable level (0.212 ng/mL), right at the lower limit of quantification (LLOQ) of the assay. No metabolite (SEQ ID NO: 10) was detected in the plasma from any subject. No PK parameters (apart from the observed Cₘₐₓ and Tₘₐₓ) could be calculated in the 2 subjects due to limited data points.

As for the 24-hour stool results, approximately 3 percent of a single dose of linaclotide was recovered on average following 7 days of dosing at 300 ug per day under both fed and fasted conditions. The recovery was predominately in the form of a major linaclotide metabolite.

### CONCLUSIONS OF STUDY

In this study of healthy subjects, the PD effects of linaclotide after 7 days of dosing were not demonstrated to be equivalent following dosing in the presence of a high-fat meal versus dosing in the fasted condition. Thus, taking linaclotide with food appears to increase the PD effects of linaclotide.

Without further description, it is believed that one of ordinary skill in the art can, using the preceding description and the illustrative examples, make and utilize the present invention and practice the claimed methods. It should be understood that the foregoing discussion and examples merely present a detailed description of certain preferred embodiments. It will be apparent to those of ordinary skill in the art that various modifications and equivalents can be made without departing from the spirit and scope of the invention. All the patents and patent publications discussed or cited above are herein incorporated by reference.

1-211. (canceled)

212. A method of decreasing the pharmacodynamic effect of a GC-C receptor agonist polypeptide formulation which is administered to a subject in need of such treatment, comprising administering the GC-C receptor agonist polypeptide formulation to the subject before the ingestion of food, wherein the GC-C receptor agonist formulation comprises a GC-C receptor agonist polypeptide, a pharmaceutically acceptable carrier, and one or more agents selected from a cation selected from Mg²⁺, Ca²⁺, Zn²⁺, Mn²⁺, K⁺, Na⁺ and Al³⁺ and a sterically hindered primary amine.

213. The method of claim 212, wherein the GC-C receptor agonist polypeptide formulation is administered to the subject 15 minutes to 4 hours before the ingestion of food, 1 to 18 hours before the ingestion of food, 4 to 12 hours before the ingestion of food, 30 minutes to 8 hours before the ingestion of food, or administered to the subject having an empty stomach.

214. The method of claim 212, wherein the subject in need of such treatment is suffering from a disorder selected from the group consisting of gastrointestinal motility disorder, chronic intestinal pseudo-obstruction, colonic pseudo-obstruction, duodenogastric reflux, dyspepsia, functional dyspepsia, nonulcer dyspepsia, functional gastrointestinal disorder, functional heartburn, gastroesophageal reflux disease (GERD), gastroparesis, irritable bowel syndrome (IBS), post-operative ileus, and constipation.

215. The method of claim 214, wherein said disorder is IBS, and said IBS is constipation-predominant IBS (IBS-c) or alternating IBS (IBS-a).

216. The method of claim 214, wherein said disorder is constipation, and said constipation is chronic constipation, idiopathic constipation, post-operative ileus, or constipation caused by opiate use.

217. The method of claim 212, wherein the pharmacodynamic effect is measured by the Bristol Stool Form Scale (BSFS), the number of spontaneous bowel movements (SBM) in a given time period or the number of complete SBM (CSBM) in a given time period.

218. The method of claim 212, wherein the pharmacodynamic effect results in a lower score on the Bristol Stool Form Scale (BSFS), fewer spontaneous bowel movements (SBM), or fewer complete spontaneous bowel movements (CSBM) when the formulation is administered to the subject before ingestion of food as compared to when the formulation is administered to the subject with food or shortly after ingestion of food.

219. The method of claim 212, wherein said polypeptide is selected from the group consisting of CCEFCCNPACTGCY (SEQ ID NO: 2), CCEFCCNPACTGC (SEQ ID NO: 3), CCEICCNPACTGCY (SEQ ID NO: 4), CCEICCNPACTGC (SEQ ID NO: 5), CCELCCNPACTGCY (SEQ ID NO: 6), CCELCCNPACTGC (SEQ ID NO: 7), CCEWCCNPACTGCY (SEQ ID NO: 8), CCEWCCNPACTGC (SEQ ID NO: 9), CCEYCCNPACTGC (SEQ ID NO: 10), PGTCEICAYAACTGC (SEQ ID NO: 11), NDDCELCVNVACTGCL (SEQ ID NO: 12), and CCEYCCNPACTGCY (SEQ ID NO: 14).

220. The method according to claim 212, wherein the cation Mg²⁺, Ca²⁺, Zn²⁺, Mn²⁺, K⁺, Na⁺ or Al³⁺ is provided as magnesium acetate, magnesium chloride, magnesium phosphate, magnesium sulfate, calcium acetate, calcium chloride, calcium phosphate, calcium carbonate, calcium sulfate, zinc acetate, zinc chloride, zinc phosphate, zinc sulfate, manganese acetate, manganese chloride, manganese phosphate, manganese sulfate, potassium acetate, potassium chloride, potassium phosphate, potassium sulfate, sodium acetate, sodium chloride, sodium phosphate, sodium sulfate, aluminum acetate, aluminum chloride, aluminum phosphate or aluminum sulfate.

221. The method according to claim 212, wherein the sterically hindered primary amine is selected from an amino acid or a compound of the formula: 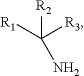
wherein R₁, R₂ and R₃ are independently selected from: H; —C(O)OH; C₁-C₆ alkyl, optionally substituted by —CO₂H, —CONH₂, or a 5-10 membered aryl or heteroaryl; C₁-C₆ alkoxyalkyl; or C₁-C₆ thioalkoxyalkyl, wherein any of the alkyl or aryl groups above can be singly or multiply substituted with halogen or —NH₂, and provided that no more than two of R₁, R₂ and R₃ are H.

222. The method according to claim 221, wherein the sterically hindered primary amine is a naturally-occurring amino acid selected from the group consisting of histidine, phenylalanine, alanine, glutamic acid, aspartic acid, glutamine, leucine, methionine, asparagine, tyrosine, threonine, isoleucine, tryptophan and valine.

223. The method according to claim 222, wherein the sterically hindered primary amine is leucine and the molar ratio of leucine to said polypeptide is at least 10:1, 20:1, or 30:1.

224. The method according to claim 221, wherein the sterically hindered primary amine is a non-naturally occurring amino acid selected from 1-aminocyclohexane carboxylic acid, cyclohexylamine, and 2-methylbutylamine.

225. The method according to claim 221, wherein the sterically hindered primary amine is chitosan.

226. The method according to claim 212, wherein the formulation comprises a cation selected from Mg²⁺, Ca²⁺, Zn²⁺, Mn²⁺, K⁺, Na⁺ or Al³⁺ and a sterically hindered primary amine.

227. The method according to claim 226, wherein the cation is Ca²⁺ and the sterically hindered primary amine is leucine, and the molar ratio of Ca²⁺ to leucine is at least 1:1, 1.5:1, or 2:1.

228. The method according to claim 226, wherein the molar ratio of cation:sterically hindered primary amine:polypeptide is 40-100:20-50:1.

229. The method according to claim 228, wherein the cation is Ca²⁺, the sterically hindered primary amine is leucine, and the molar ratio of Ca²⁺:leucine:polypeptide is 100:30:1, 80:40:1, 80:30:1, 80:20:1, 60:30:1, 60:20:1, 50:30:1, 50:20:1, 40:20:1, 20:20:1, 10:10:1, 10:5:1, 5:10:1 or 5:5:1.

230. The method according to claim 229, wherein the molar ratio of Ca²⁺:leucine:polypeptide is 60:30:1.

231. The method according to claim 212, wherein the formulation further comprises one or more of a pharmaceutically acceptable binder, a pharmaceutically acceptable glidant, lubricant or additive that acts as both a glidant and lubricant, an antioxidant, or a pharmaceutically acceptable filler.

232. The method according to claim 231, wherein
the antioxidant, when present, is BHA, vitamin E or propyl gallate;
the pharmaceutically acceptable binder, when present, is selected from polyvinyl alcohol, polyvinylpyrrolidone (povidone), a starch, maltodextrin or a cellulose ether; and
the pharmaceutically acceptable filler, when present, is selected from cellulose, isomalt, mannitol or dibasic calcium phosphate.

233. The method according to claim 232, wherein the cellulose ether, when present, is selected from methylcellulose, ethylcellulose, carboxymethylcellulose, hydroxyethyl cellulose, hydroxyethyl methylcellulose, hydroxypropyl cellulose and hydroxypropyl methylcellulose; and
the cellulose, when present, is selected from microfine cellulose and microcrystalline cellulose.

234. The method according to claim 231, wherein the pharmaceutically acceptable filler comprises particles having an average diameter between 150 μm and 1000 μm.

235. The method according to claim 231, wherein the formulation comprises a pharmaceutically acceptable filler and the weight ratio of the polypeptide to pharmaceutically acceptable filler is between 1:25 and 1:2,500; between 1:100 and 1:2000; or between 1:100 and 1:1000.

236. The method of claim 212, wherein the GC-C receptor antagonist formulation is in the form of a capsule or tablet that comprises 50 μg to 1 mg GC-C receptor agonist polypeptide.

237. The method of claim 236, wherein the capsule or tablet is comprises 100 μg, 150 μg, 200 ug, 300 μg, 400 μg, 500 μg or 600 μg GC-C receptor agonist polypeptide.

238. A method of decreasing the pharmacodynamic effect of a GC-C receptor agonist polypeptide formulation which is administered to a subject suffering from irritable bowel syndrome or constipation, comprising administering the GC-C receptor agonist polypeptide formulation to the subject before the ingestion of food, wherein the GC-C receptor agonist formulation comprises a GC-C receptor agonist polypeptide, a pharmaceutically acceptable carrier, Ca⁺² provided as calcium chloride, and leucine.

239. A method of decreasing the pharmacodynamic effect of a linaclotide formulation which is administered to a subject suffering from irritable bowel syndrome or constipation, comprising administering the GC-C receptor agonist polypeptide formulation to the subject before the ingestion of food, wherein the GC-C receptor agonist formulation comprises linaclotide or a pharmaceutically acceptable salt of linaclotide, a pharmaceutically acceptable carrier, Ca⁺² provided as calcium chloride, and leucine.

240. A method of decreasing the pharmacodynamic effect of a GC-C receptor agonist polypeptide which is administered to a subject in need of such treatment, comprising administering the GC-C receptor agonist polypeptide to the subject before the ingestion of food.

241. The method of claim 240, wherein the GC-C receptor agonist polypeptide is linaclotide.

242. The method of claim 240, wherein the GC-C receptor agonist polypeptide formulation is administered to the subject 15 minutes to 4 hours before the ingestion of food, 1 to 18 hours before the ingestion of food, 4 to 12 hours before the ingestion of food, 30 minutes to 8 hours before the ingestion of food, or administered to the subject having an empty stomach.

243. The method of claim 240, wherein the GC-C receptor agonist polypeptide formulation is administered to the subject at least 15 minutes before the ingestion of food, at least 30 minutes before the ingestion of food, at least 1 hour before the ingestion of food, or at least 2 hours before the ingestion of food.

244. The method of claim 240, wherein the GC-C receptor agonist polypeptide formulation is administered to the subject at least 4 hours after the ingestion of food, at least 6 hours after the ingestion of food, at least 8 hours after the ingestion of food, or at least 10 hours after the ingestion of food.

245. The method according to claim 240, wherein said GC-C receptor agonist polypeptide is administered as a formulation comprising the GC-C receptor agonist polypeptide and a pharmaceutically acceptable excipient.

246. The method according to claim 240, wherein the subject in need of such treatment is suffering from a disorder selected from the group consisting of irritable bowel syndrome (IBS) and constipation.

247. The method of claim 246, wherein said disorder is IBS, and said IBS is constipation-predominant IBS (IBS-c) or alternating IBS (IBS-a).

248. The method of claim 246, wherein said disorder is constipation, and said constipation is chronic constipation, idiopathic constipation, post-operative ileus, or constipation caused by opiate use.

249. A method of increasing the pharmacodynamic effect of a GC-C receptor agonist polypeptide which is administered to a subject in need of such treatment, comprising administering the GC-C receptor agonist polypeptide to the subject with the ingestion of food or within two hours after the ingestion of food.

250. The method according to claim 249, wherein the GC-C receptor agonist polypeptide is administered with a meal, within two hours after the ingestion of food, within one hour after the ingestion of food, within 30 minutes after the ingestion of food, or within 15 minutes after the ingestion of food.

251. The method of any one of claims 249, wherein the GC-C receptor agonist polypeptide is linaclotide.

252. A method of treating irritable bowel syndrome or constipation in a subject in need of such treatment, comprising administering a GC-C receptor agonist polypeptide to the subject before the ingestion of food.

253. The method of claim 252, wherein GC-C receptor agonist polypeptide formulation is administered to the subject selected from at least 15 minutes before the ingestion of food, at least 30 minutes before the ingestion of food, or having an empty stomach.

254. The method of claim 252, wherein said disorder is IBS, and said IBS is constipation-predominant IBS (IBS-c) or alternating IBS (IBS-a).

255. The method of claim 252, wherein said disorder is constipation, and said constipation is chronic constipation, idiopathic constipation, post-operative ileus, or constipation caused by opiate use.

256. The method of claim 252, wherein said polypeptide is selected from the group consisting CCEFCCNPACTGCY (SEQ ID NO: 2), CCEFCCNPACTGC (SEQ ID NO: 3), CCEICCNPACTGCY (SEQ ID NO: 4), CCEICCNPACTGC (SEQ ID NO: 5), CCELCCNPACTGCY (SEQ ID NO: 6), CCELCCNPACTGC (SEQ ID NO: 7), CCEWCCNPACTGCY (SEQ ID NO: 8), CCEWCCNPACTGC (SEQ ID NO: 9), CCEYCCNPACTGC (SEQ ID NO: 10), PGTCEICAYAACTGC (SEQ ID NO: 11), NDDCELCVNVACTGCL (SEQ ID NO: 12), and CCEYCCNPACTGCY (SEQ ID NO: 14).

257. The method of claim 256, wherein the GC-C receptor agonist is administered as a formulation comprising the GC-C receptor agonist polypeptide, a pharmaceutically acceptable carrier, and one or more agents selected from a cation selected from Mg²⁺, Ca²⁺, Zn²⁺, Mn²⁺, K⁺, Na⁺ and Al³⁺ or a sterically hindered primary amine.
